(19) United States
(12) Patent Application Publication
(10) Pub. No.: US 2010/0034780 A1
(43) Pub. Date: Feb. 11, 2010

(54) TUMOR ANTIGEN PEPTIDE DERIVED FROM AMACR

(76) Inventors: Noriyuki Sato, Sapporo-Shi (JP); Toshihiko Torigoe, Sapporo-Shi (JP); Ichiya Honma, Sapporo-Shi (JP); Yoshihiko Hirohashi, Sapporo-Shi (JP); Kenji Harada, Osaka-Shi (JP)

Correspondence Address: BIRCH STEWART KOLASCH & BIRCH, PO BOX 747, FALLS CHURCH, VA, 22040-0747, US

(21) Appl. No.: 12/096,572

(22) PCT Filed: Jun. 27, 2006
(86) PCT No.: PCT/JP2006/312788

§ 371 Date: Jul. 11, 2008

(30) Foreign Application Priority Data
Dec. 8, 2005 (JP) 2005-354263

(51) Int. Cl. A61K 38/43; A61K 35/00; C12N 9/00; C12N 5/0784; C12N 5/0783; C12N 5/00; A61K 48/00; C07K 16/40; C07H 21/00

(52) U.S. Cl. 424/93.7; 424/94.1; 435/183; 435/325; 435/372.3; 435/375; 514/44 R; 530/387.9; 536/23.2

(57) ABSTRACT

The present invention relates to a peptide which comprises a partial peptide derived from AMACR and is capable of binding to an HLA antigen and is recognized by a CTL, and a pharmaceutical composition comprising the peptide and a pharmaceutically acceptable carrier, and the like.

### TECHNICAL FIELD

The invention relates to use of alpha-methylacyl-CoA racemase (referred as AMACR below) and a peptide derived therefrom in the field of cancer immunity.

### BACKGROUND ART

Cancer therapy can be roughly classified into “local therapy” and “systemic therapy”. The local therapy includes “surgical therapy” and “radiation therapy”. The systemic therapy is mainly medicinal therapy with medicaments such as anti-cancer agents and hormonal agents which are administered, for example, orally or intravenously. At present, 40% of patients are cured by the local therapy and 7-10% by involving anti-cancer agents in some way. In other words, approximately 50% of patients can be cured by surgical, radiation or chemical therapy, but the remaining 50% are not. To treat such cancer patients in future, two actions have been mainly considered to be necessary.

One is to increase the ratio of patients cured by the local therapy through early detection and the other is to develop novel therapies by which the ratio cured by the systemic therapy will increase.

As a novel therapy, many molecular-targeted agents, which have a new concept and do not show serious adverse effects such as those caused by the previous anti-cancer agents, e.g. myelosuppression, have recently been developed and launched. The examples of those agents include Herceptin® which is an antibody preparation targeting Her2 molecule highly expressed in breast cancer, and Iressa® which is an inhibitor of tyrosine kinase of epidermal growth factor receptor (EGF-R) highly expressed in lung cancer. The anti-tumor effects of those agents have been reported to be strong and significant.

There is no molecular-targeted agent satisfied enough, however. Regarding molecular targeted agents, other serious side effects including interstitial lung disease than those observed with previous anti-cancer agents such as myelosuppression have been reported. Therefore, research and development of an agent targeting a novel cancer-specific molecule or a novel therapeutic method has been required.

Under the conditions, cancer immunotherapy utilizing immunity of a living body has been intensively studied and developed in recent years. It is known that immune system, particularly cytotoxic T cells (CTLs) which recognize tumor cells in the body, play a significant role in vivo rejection of tumor cells. CTLs recognize a complex between a peptide, that is, a tumor antigen peptide and a major histocompatibility complex class I antigen (MHC class I antigen, which is referred to as “HLA antigen” in the case of human) on the cell surface of tumor cells by T cell receptor (TCR), and attack the cells.

By using such a tumor antigen peptide, a tumor antigen protein from which the tumor antigen peptide is derived, or a nucleic acid encoding the same as a cancer vaccine, treatment based on the induction and enhancement of tumor-specific CTLs in the body of a tumor patient has become possible.

As a tumor antigen protein, T. Boon et al. identified a protein named MAGE from human melanoma cells for the first time in 1991 (see, Non Patent Literature 1). Subsequently, several additional tumor antigen proteins have been identified mainly from melanoma cells.

In order to apply tumor antigen proteins and peptides to tumor therapy or diagnostics, it is necessary to identify novel ones which can be applied widely, for example to adenocarcinoma which occurs much more frequently than melanoma.

α-Methylacyl-CoA-racemase (AMACR) is an enzyme involved in β-oxidation of branched fatty acids and bile acid intermediates and known to be specifically expressed and up-regulated in epithelial cells in prostate cancer.

An increase of level of an AMACR-specific antibody in blood in prostate cancer patients has been observed (see, Non Patent Literature 2). Further, cell proliferation of a prostate cancer cell line showing high expression of AMACR was decreased when the gene expression of AMACR was attenuated by AMACR-specific siRNA, which suggests that AMACR also has an important role in regulation of cell proliferation of the prostate cancer cell line (see, Non Patent Literature 3). In addition, it has been reported that adult-onset sensory motor neuropathy was caused by mutation in AMACR gene (see, Non patent Literature 4). It has not known, however, that AMACR is a tumor antigen protein useful as a cancer vaccine.
Non Patent Literature 1: Science, 254, 1643-1647(1991)
Non Patent Literature 2: J. Natl. Cancer Inst., 96(11), 834-843(2004)
Non Patent Literature 3: Cancer Res., 63(21), 7365-7376(2003)
Non Patent Literature 4: Nat. Genet., 24(2), 188-191(2000)

### DISCLOSURE OF THE INVENTION

### Problem To Be Solved By the Invention

An object of the invention is to provide use of AMACR and a peptide derived therefrom in the field of cancer immunity, and the like.

### Means For Solving the Problem

The inventors synthesized peptides which were potential to bind to HLA-A24 based on the amino acid sequence of human AMACR and identified those having a high binding affinity. When peripheral blood T lymphocytes of HLA-A24 positive prostate cancer patients were stimulated with the AMACR peptide in an attempt to induce CTLs, a CTL specific to the peptide was induced. The result demonstrates that AMACR can be a target antigen in cancer immunotherapy (tumor antigen protein) against prostate cancer and those peptides are useful as a tumor antigen peptide in vaccine therapy for prostate cancer patients.

AMACR is known to show an increased expression not only in prostate cancer but in different cancer tissues (Am J Surg Pathol., 26,926-931(2002), Hum Pathol., 34,792-796(2003), Am J Surg Pathol., 29(3), 381-389(2005), Appl Immunohistochem Mol Morphol 13,252-255(2005)). From the tact that AMACR is a tumor antigen protein, which has been revealed by the present invention as mentioned above, the tumor antigen protein AMACR and the AMACR-derived tumor antigen peptide of the present invention are considered to be useful for a variety of cancers showing increased expression of AMACR as prostate cancer (for example, bowel cancer, ovarian cancer, bladder cancer, lung cancer, renal cell cancer, lymphoma, melanoma, liver cancer, gastric cancer, pancreas cancer or uterine cancer).

The present invention has been accomplished on the basis of the aforementioned findings.

Accordingly, the present invention relates to the followings:

(1) A peptide which comprises a partial peptide derived from AMACR and is capable of binding to an HLA antigen and is recognized by a CTL;

(2) The peptide of (1) above, wherein the HLA antigen is HLA-A 24 or HLA-A2 antigen;

(3) The peptide of (2) above, which comprises the amino acid sequence of any one of SEQ ID NOS: 3 to 33;

(4) The peptide of (3) above, which consists of the amino acid sequence of any one of SEQ ID NOS: 3 to 5;

(5) The peptide of (4) above, which consists of the amino acid sequence of SEQ ID NO: 5;

(6) A peptide which comprises an amino acid sequence which is the same as the amino acid sequence of any one of SEQ ID NOS: 3 to 23 except that the amino acid at position 2 is substituted by tyrosine, phenylalanine, methionine or tryptophan, and/or the C terminal amino acid by phenylalanine, leucine, isoleucine, tryptophan or methionine, and is capable of binding to HLA-A24 antigen and is recognized by a CTL;

(7) A peptide which consists of an amino acid sequence which is the same as the amino acid sequence of any one of SEQ ID NOS: 3 to 5 except that the amino acid at position 2 is substituted by tyrosine, phenylalanine, methionine or tryptophan, and/or the C terminal amino acid by phenylalanine, leucine, isoleucine, tryptophan or methionine, and is capable of binding to HLA-A24 antigen and is recognized by a CTL;

(8) A peptide which consists of an amino acid sequence which is the same as the amino acid sequence of SEQ ID NO: 5 except that the amino acid at position 2 is substituted by tyrosine, phenylalanine, methionine or tryptophan, and/or the C terminal amino acid by phenylalanine, leucine, isoleucine, tryptophan or methionine, and is capable of binding to HLA-A24 antigen and is recognized by a CTL;

(9) A peptide which comprises an amino acid sequence which is the same as the amino acid sequence of any one of SEQ ID NOS: 24 to 33 except that the amino acid at position 2 is substituted by leucine, methionine, valine, isoleucine or glutamine and/or the C terminal amino acid by valine or leucine, and is capable of binding to HLA-A2 antigen and is recognized by a CTL;

(10) An epitope peptide which comprises the peptide of any one of (1) to (9) above;

(11) A pharmaceutical composition which comprises the peptide of any one of (1) to (10) above and a pharmaceutically acceptable carrier;

(12) A nucleic acid which comprises a polynucleotide encoding the peptide of any one of (1) to (10) above;

(13) A pharmaceutical composition which comprises the nucleic acid of (12) above and a pharmaceutically acceptable carrier;

(14) A pharmaceutical composition which comprises AMACR and a pharmaceutically acceptable carrier;

(15) The pharmaceutical composition of (14) above, wherein AMACR has the amino acid sequence of SEQ ID NO: 2;

(16) A pharmaceutical composition which comprises a nucleic acid comprising a polynucleotide encoding AMACR and a pharmaceutically acceptable carrier;

(17) The pharmaceutical composition of (16) above, wherein the polynucleotide encoding AMACR comprises the base sequence of SEQ ID NO: 1, or encodes the amino acid sequence of SEQ ID NO: 2;

(18) A method of preparing an antigen presenting cell, wherein a cell having antigen-presenting ability is brought into contact in vitro with any one of:
(a) the peptide of any one of (1) to (10) above,
(b) the nucleic acid of (12) above,
(c) AMACR, and
(d) a nucleic acid comprising a polynucleotide encoding AMACR;

(19) An antigen presenting cell prepared by the method of (18) above;

(20) A pharmaceutical composition which comprises the antigen presenting cell of (19) above and a pharmaceutically acceptable carrier;

(21) A method of inducing a CTL, wherein peripheral blood lymphocytes are brought into contact in vitro with any one of:
(a) the peptide of any one of (1) to (10) above,
(b) the nucleic acid of (12) above,
(c) AMACR, and
(d) a nucleic acid comprising a polynucleotide encoding AMACR;

(22) A CTL induced by the method of (21) above;

(23) A pharmaceutical composition which comprises the CTL of (22) above and a pharmaceutically acceptable carrier;

(24) The pharmaceutical composition of (11), (13), (14), (15), (16), (17) or (20) above, which is used as an inducer of CTL;

(25) The pharmaceutical composition of (11), (13), (14), (15), (16), (17), (20) or (23) above, which is used as a cancer vaccine;

(26) An antibody which specifically binds to the peptide of any one of (1) to (9) above;

(27) An HLA monomer, HLA dimer, HLA tetramer or HLA pentamer which comprises the peptide of any one of (1)-(9) above and an HLA antigen;

(28) A reagent for detecting a CTL specific to a AMACR-derived tumor antigen peptide, which comprises as a component the HLA monomer, HLA dimer, HLA tetramer or HLA pentamer of (27) above.

### EFFECT OF THE INVENTION

AMACR and the AMACR-derived tumor antigen peptide or the nucleic acid encoding the same of the present invention can be useful as a cancer vaccine. Further, the AMACR-derived tumor antigen peptide is also useful as a component of an HLA tetramer and the like to detect a CTL.

### BEST MODE FOR CARRYING OUT THE INVENTION

Abbreviations for amino acids, (poly)peptides, (poly)nucleotides and the like used herein, follow rules of IUPAC-IUB (IUPAC-IUB Communication on Biological Nomenclature, Eur. J. Biochem., 138: 9 (1984)), “Guidelines for preparing the specification containing a base sequence or an amino acid sequence” (Japan Patent Office), and symbols commonly used in this field.

### 1) Protein of the Present Invention AMACR

The tumor antigen protein of the present invention AMACR comprises the amino acid sequence of SEQ ID NO: 2 or an amino acid sequence similar to the aforementioned amino acid sequence. The protein AMACR may be a protein originated from natural source (e.g., a prostate cancer cell line) or a recombinant protein.

Here, the amino acid sequence of SEQ ID NO: 2 is registered with the GenBank database under Accession No. NM_{—}014324, Accession No. NP_{—}055139, and represents human AMACR (alpha-methylacyl-CoA racemase).

The “protein comprising the amino acid sequence of SEQ ID NO: 2” specifically includes a protein consisting of the amino acid sequence of SEQ ID NO: 2, and a protein consisting of an amino acid sequence which comprises the amino acid sequence of SEQ ID NO: 2 having an additional amino acid sequence(s) attached to the N and/or C terminus. “Additional amino acid sequence” may be the amino acid sequence derived from other structural genes than AMACR.

The “protein comprising an amino acid sequence similar to the amino acid sequence of SEQ ID NO: 2” specifically includes the following proteins (a) to (c):

(a) a protein comprising an amino acid sequence which is the same as the amino acid sequence of SEQ ID NO: 2 except that one or more amino acids are deleted, substituted and/or added, and having an activity as a tumor antigen protein;

(b) a protein comprising an amino acid sequence having at least 70% sequence identity with the amino acid sequence of SEQ ID NO: 2, and having an activity as a tumor antigen protein;

(c) a protein being encoded by a polynucleotide capable of hybridizing to a complementary strand of a polynucleotide encoding the amino acid sequence of SEQ ID NO: 2 under stringent conditions, and having an activity as a tumor antigen protein.

Preferred examples include a protein consisting of an amino acid sequence similar to the amino acid sequence of SEQ ID NO: 2. Examples of such a protein include the proteins (a′) to (c′) below:

(a′) a protein consisting of an amino acid sequence which is the same as the amino acid sequence of SEQ ID NO: 2 except that one or more amino acids are deleted, substituted and/or added, and having an activity as a tumor antigen protein;

(b′) a protein consisting of an amino acid sequence having at least 70% sequence identity with the amino acid sequence of SEQ ID NO: 2, and having an activity as a tumor antigen protein;

(c′) a protein being encoded by a polynucleotide capable of hybridizing to a complementary strand of a polynucleotide encoding the amino acid sequence of SEQ ID NO: 2 under stringent conditions, and having an activity as a tumor antigen protein.

The “protein comprising an amino acid sequence which is the same as the amino acid sequence of SEQ ID NO: 2 except that one or more amino acids are deleted, substituted and/or added” in (a) above refers to a protein produced artificially, that is, a modified (variant) protein, or an allele variant present in a living body, for example.

In this respect, there is no limitation regarding the number or position of modification (mutation) in the protein as far as the activity of the protein of the present invention is maintained. Criteria based on which one can determine the number or position of the amino acid residue to be deleted, substituted and/or added without reducing the activity can be obtained using a computer program well known in the art, such as DNA Star software. For example, the number of mutation would typically be within 10%, preferably 5% of the total amino acid residues. Furthermore, the amino acid introduced by substitution preferably has similar characteristics to that to be substituted in view of retention of structure, which characteristics include polarity, charge, solubility, hydrophobicity, hydrophilicity, amphipathicity, etc. For instance, Ala, Val, Leu, Ile, Pro, Met, Phe and Trp are classified into nonpolar amino acids; Gly, Ser, Thr, Cys, Tyr, Asn and Gln into non-charged amino acids; Asp and Glu into acidic amino acids; and Lys, Arg and His into basic amino acids. One of ordinary skill in the art can select an appropriate amino acid(s) falling within the same group on the basis of these criteria.

The “protein comprising an amino acid sequence having at least 70% sequence identity with the amino acid sequence of SEQ ID NO: 2” in (b) above includes a protein comprising an amino acid sequence having at least about 70%, preferably about 80%, more preferably about 90%, and further more preferably about 95% sequence identity with the amino acid sequence of SEQ ID NO: 2, and specifically, a protein consisting of a partial amino acid sequence of SEQ ID NO: 2.

The term “sequence identity” herein used refers to the identity and homology between two proteins. The “sequence identity” is determined by comparing two sequences aligned optimally over the sequence region to be compared. In this context, the optimum alignment of the proteins to be compared may have an addition or deletion (e.g., “gap”). The sequence identity can be calculated by preparing an alignment using, for example, Vector NTI, ClustalW algorithm (Nucleic Acid Res., 22 (22): 4673-4680(1994)). The sequence identity can be determined using software for sequence analysis, specifically, Vector NTI or GENETYX-MAC, or a sequencing tool provided by a public database. Such a public database is commonly available at Web site (http://www.ddbj.nig.ac.ip).

The “polynucleotide capable of hybridizing to a complementary strand of a polynucleotide encoding the amino acid sequence of SEQ ID NO: 2 under stringent conditions” in (c) above includes a polynucleotide comprising a base sequences having at least about 40%, preferably about 60%, more preferably about 70%, still more preferably about 80%, further more preferably about 90%, and most preferably about 95% sequence identity with a polynucleotide encoding the amino acid sequence of SEQ ID NO: 2. Specifically, a polynucleotide comprising a base sequence having at least about 40%, preferably about 60%, more preferably about 70%, still more preferably about 80%, further more preferably about 90%, and most preferably about 95% sequence identity with the base sequence of SEQ ID NO: 1 is exemplified. More specifically, a polynucleotide consisting of a partial sequence of the base sequence of SEQ ID NO: 1 is exemplified.

Hybridization can be conducted according to a method known per se or a method equivalent thereto, for example, that described in a fundamental text book such as “Molecular Cloning 2nd Edt. Cold Spring Harbor Laboratory Press (1989)”, and the like. Also, it can be performed using a commercially available library according to the instructions attached thereto.

The “stringent conditions” herein used can be determined on the basis of the melting temperature (Tm) of nucleic acids forming a complex or a nucleic acid binding to a probe as described in literatures (Berger and Kimmel, 1987, “Guide to Molecular Cloning Techniques Methods in Enzymology”, Vol. 152, Academic Press, San Diego Calif.; or “Molecular Cloning” 2nd Edt. Cold Spring Harbor Laboratory Press (1989), ibid.).

For example, hybridization can be carried out in a solution containing 6×SSC (20×SSC means 333 mM sodium citrate, 333 mM NaCl), 0.5% SDS and 50% formamide at 42° C., or in a solution containing 6×SSC (without 50% formamide) at 65° C.

Washing after the hybridization can be conducted under a condition around “1×SSC, 0.1% SDS, 37° C.”. The complementary strand preferably remains to be bound to the target sense strand when washed under such washing conditions. More stringent hybridization conditions may involve washing conditions of around “0.5×SSC, 0.1% SDS, 42° C.” and still more stringent hybridization conditions around “0.1×SSC, 0.1% SDS, 65° C.” although it is not limited thereto.

The protein consisting of an amino acid sequence similar to the amino acid sequence of SEQ ID NO: 2 specifically includes the splicing variants of human AMACR of GenBank Accession No. NM203382 and Accession No. NP976316.

The protein of the present invention AMACR has an activity as a tumor antigen protein. The term “activity as a tumor antigen protein” refers to an activity detected by a conventional assay for the activity of a tumor antigen protein. Specifically, it refers to the characteristics that a cell expressing AMACR is recognized by a CTL, that is, the cell exhibits reactivity to a CTL, in other words, the protein of the present invention or antigen peptides derived therefrom activates or induces a CTL.

In this respect, the “cell” preferably expresses an HLA antigen. Accordingly, the “activity as a tumor antigen protein” more specifically refers to the characteristics that, when the protein of the present invention is expressed in a cell expressing an HLA antigen such as HLA-A24 or HTA-A2, a complex between a tumor antigen peptide originated from the protein of the present invention and the HLA antigen is presented on the cell surface and consequently the cell is recognized by a CTL, in other words, a CTL is activated (induced).

The characteristics of the protein of the present invention as mentioned above can be easily determined by a known method or a method equivalent thereto, such as ⁵¹Cr release assay (J. Immunol., 159: 4753, 1997), LDH release assay using LDH Cytotoxicity Detection Kit (Takara Bio, Inc.), measurement of cytokines, and the like. The detailed protocol of assay will hereinafter be illustrated.

First, a host cell such as 293-EBNA cell (Invitrogen) is co-transfected with an expression vector comprising a DNA encoding the protein of the present invention and an expression vector comprising a DNA encoding an HLA antigen. The DNA encoding an HLA antigen includes a DNA encoding HLA-A24 antigen or HLA-A2 antigen. Examples of a DNA encoding HLA-A24 antigen include HLA-A2402 cDNA (Cancer Res., 55: 4248-4252 (1995), Genbank Accession No. M64740). Examples of DNA encoding HLA-A2 antigen include HLA-A0201 cDNA (GenBank Acc. No. M84379).

The transfection as mentioned above can be conducted by Lipofectin method using lipofectamine reagent (GIBCO BRL), and the like. Then, a CTL restricted to the HLA antigen used is added and allowed to react, followed by measurement of various cytokines (for example, IFN-γ) produced by the activated (reacting) CTL by a method such as ELISA, for example. The CTL usable herein may be prepared by stimulating peripheral blood lymphocytes with the protein of the present invention AMACR or established according to the method of Int. J. Cancer, 39, 390-396, 1987, N. Eng. J. Med, 333, 1038-1044, 1995, or the like.

The CTL-inducing activity of the protein of the present invention can also be examined in vivo by an assay where a model animal for human is used (WO 02/47474; Int. J. Cancer. 100, 565-570 (2002).

The protein of the present invention can be prepared by a method known per se that is used for purifying a protein from natural products (e.g., a prostate cancer cell line) or by a method hereinafter described comprising culturing a transformant carrying a nucleic acid comprising a polynucleotide encoding the protein of the present invention.

### 2) AMACR-Derived Peptide of the Present Invention

The AMACR-derived peptide of the present invention, which may be referred to as “peptide of the present invention”, is a tumor antigen peptide which comprises a partial peptide of the protein of the present invention AMACR and is capable of binding to an HLA antigen and is recognized by a CTL. Thus, the peptide of the present invention may comprise a peptide corresponding to any position of the amino acid sequence of the protein of the present invention AMACR and being of any length, as long as the peptide comprises a part of the amino acid sequence of the protein of the present invention AMACR as defined above and can form a complex with an HLA antigen that is recognized by a CTL.

The peptide of the present invention can be identified by synthesizing a candidate peptide, which is a partial fragment of AMACR, and subjecting the candidate peptide to an assay to examine whether or not a CTL recognizes a complex between the candidate peptide and an HLA antigen, that is, whether or not the candidate peptide has the activity as a tumor antigen peptide.

Synthesis of the peptide can be conducted according to a method generally used in the field of peptide chemistry. Such a method can be found in literatures including Peptide Synthesis, Interscience, New York, 1966; The Proteins, Vol. 2, Academic Press Inc., New York, 1976; Peptide-Gosei, Maruzen, Inc., 1975; Peptide-Gosei no Kiso to Jikken, Maruzen, Inc., 1985; and Iyakuhin no Kaihatsu (Zoku), Vol. 14, Peptide-Gosei, Hirokawa-syoten, 1991.

The method for identification of the tumor antigen peptide of the present invention will hereinafter be described in detail.

The regularity (motif) in an amino acid sequence of a tumor antigen peptide that binds to an HLA antigen to be presented has been elucidated in relation to some HLA types such as HLA-A1, -A0201, -A0204, -A0205, -A0206, -A0207, -A11, -A24, -A31, -A6801, -B7, -B8, -B2705, -B37, -Cw0401, and -Cw0602. See, Immunogenetics, 41: p. 178, 1995, etc. For example, the motifs for HLA-A24 are known to have an amino acid sequence of 8 to 11 amino acids, wherein the amino acid at position 2 is tyrosine, phenylalanine, methionine or tryptophan, and the C-terminal amino acid phenylalanine, leucine, isoleucine, tryptophan or methionine (J. Immunol., 152, p 3913, 1994, Immunogenetics, 41: p 178, 1995. J. Immunol., 155:p 4307, 1994). As for motifs for HLA-A2, those listed in Table 1 are known (Immunogenetics, 41, p 178, 1995, J. Immunol., 155: p 4749, 1995).

TABLE 1

| | Amino acid | |
|---|---|---|
| | at position 2 | Amino acid |
| HLA-A2 type | from N-terminus | at C-terminus |
| HLA-A0201 | L, M | V, L |
| HLA-A0204 | L | L |
| HLA-A0205 | V, L, I, M | L |
| HLA-A0206 | V, Q | V, L |
| HLA-A0207 | L | L |

* All the peptides are 8 to 11 amino acids in length.

Recently, it has become possible to search a peptide sequence expected to be capable of binding to an HLA antigen via the internet using BIMAS software; NIH (http://bimas.dcrt.nih.gov/molbio/hla_bind/).

As for the length of the peptide, analysis of antigen peptides binding to various HLA molecules revealed that it is generally about 8 to 14 amino acids (Immunogenetics, 41: 178, 1995). However, in the cases of HLA-DR, -DP, and -DQ, peptides consist of 14 amino acids or more are known.

It is easy to select a portion corresponding to the peptide from the amino acid sequence of AMACR of the present invention considering the motif. For example, a sequence expected to be capable of binding to an HLA antigen may be easily selected by means of BIMAS software. The peptide of the present invention can be identified by synthesizing the selected candidate peptide by the above-mentioned method, and examining whether or not the candidate peptide binds to an HLA antigen and is recognized by a CTL, that is, whether or not the candidate peptide has an activity as a tumor antigen peptide.

Specifically, identification can be done by the method described in J. Immunol., 154, p 2257, 1995. Thus, a candidate peptide is added to stimulate in vitro peripheral blood lymphocytes isolated from a human subject positive for an HLA antigen which is expected to present the candidate peptide. When a CTL specifically recognizing the HLA-positive cell pulsed with the candidate peptide is induced, the candidate peptide is possibly a tumor antigen peptide. Whether or not the induction of CTL occurs may be examined by, for example, measuring the amount of various cytokines (e.g., IFN-γ) produced by the CTL in response to the antigen-presenting cell using ELISA or the like. Alternatively, the induction of CTL can also be examined by ⁵¹Cr release assay wherein the cytotoxicity of a CTL against an antigen-presenting cell labeled with ⁵¹Cr is measured (Int. J. Cancer, 58: p 317, 1994).

Furthermore, the induction of CTL can be examined by pulsing a cell such as 293-EBNA cell (Invitrogen) with a candidate peptide, wherein the cell has been introduced with an expression plasmid for cDNA encoding a type of HLA antigens expected to present the candidate peptide, reacting the cell with a CTL restricted to the HLA antigen of the aforementioned type that is expected to present the the candidate peptide, and measuring various cytokines (e.g., IFN-γ) produced by the CTL (J. Exp. Med., 187: 277, 1998).

Examples of the HLA antigen include HLA-A24 antigen and HLA-A2 antigen. To select an HLA-A24-restricted tumor antigen peptide, HLA-A2402 cDNA (Cancer Res., 55: 4248-4252 (1995), Genbank Accession No. M64740) can be used as the cDNA encoding the HLA antigen. To select an HLA-A2-restricted tumor antigen peptide, HLA-A0201 cDNA (GenBank Acc. No. M84379) can be used as the cDNA encoding HLA antigen.

As for CTLs, in addition to those obtained by stimulating human peripheral blood lymphocytes with a peptide, CTLs established by a method described in literatures (Int. J. Cancer, 39, 390-396, 1987; N. Eng. J. Med, 333, 1038-1044, 1995) may be used.

The in vivo activity of the peptide of the present invention can be determined by an assay which uses an animal model for human (WO 02/47474, Int J. Cancer 100, 565-570 (2002)).

In the above case, the regularity (motif) of the sequence of a tumor antigen peptide is known; however, when the motif of a peptide is unknown, as is the case for HLA-B55 or HLA-A26, the tumor antigen peptide of the present invention can be identified according to the method described in, for example, WO97/46676, only if a CTL cell line capable of recognizing a complex between the HLA antigen and a tumor antigen peptide is available.

Specific examples of the peptide of the present invention include a partial peptide derived from AMACR consisting of the amino acid sequence of SEQ ID NO: 2, and being capable of binding to an HLA antigen and being recognized by a CTL. Preferred examples include a peptide capable of binding to HLA-A24 or HLA-A2 antigen, considering the HLA antigen to which the peptide of the present invention binds. The length of the peptide may be preferably 8 to 14 amino acids, more preferably 8 to 11 amino acids.

Specifically, the peptide of the present invention includes a peptide comprising the amino acid sequence of any one of SEQ ID NOS: 3 to 33 and being capable of binding to an HLA antigen and being recognized by a CTL. The length of the peptide may be preferably 9 to 14 amino acids, more preferably 9 to 11 amino acids. More specifically, as an HLA-A24-binding tumor antigen peptide, a peptide consisting of any one of the amino acid sequences of SEQ ID NOS: 3 to 23, being capable of binding to an HLA antigen and being recognized by a CTL (Table 2 below) is exemplified. Preferably, a peptide consisting of the amino acid sequence of SEQ ID NO: 3, 4 or 5 is exemplified.

Further, as an HLA-A2-binding tumor antigen peptide, a peptide consisting of any one of the amino acid sequences of SEQ ID NOS: 24 to 33, being capable of binding to an HLA antigen and being recognized by a CTL is exemplified (Table 3 below).

In the scope of the present invention, the peptide of the present invention includes not only a peptide consisting of a part of the amino acid sequence of SEQ ID NO: 2 but also a variant (modified) peptide produced by partly modifying the aforementioned peptide, provided that the variant peptide has characteristics of being capable of binding to an HLA antigen and being recognized by a CTL. Specifically, a variant peptide comprising an amino acid sequence which is the same as the amino acid sequence of the peptide of the present invention consisting of a part of the amino acid sequence of AMACR, specifically that of SEQ ID NO: 2, except that at least one amino acid modification has been introduced, and having an activity as a tumor antigen peptide, i.e. being capable of binding to an HLA antigen and being recognized by a CTL, falls within the scope of the present invention.

The “modification” of an amino acid residue means substitution, deletion and/or addition of an amino acid residue including addition to the N- and/or C-terminus of peptide, and is preferably substitution of an amino acid residue. When the modification involves amino acid substitution, the number or position of the amino acid to be substituted can be selected arbitrarily as far as an activity as a tumor antigen peptide is maintained; however, it is preferred that the substitution involves 1 to several amino acids since tumor antigen peptides are generally about 8-14 amino acids in length as mentioned above.

The variant peptide of the present invention is preferably 8 to 14 amino acids in length (in the cases of HLA-DR, -DP, or -DQ, however, peptides consisting of 14 amino acids or more are acceptable).

As mentioned above, the motif in an antigen peptide that binds to an HLA antigen and is presented is known in regard to certain HLA types, such as HLA-A1, -A0201, -A0204, -A0205, -A0206, -A0207, -A11, -A24, -A31, -A6801, -B7, -B8, -B2705, -B37, -Cw0401 and -Cw0602. Further, it is possible to search for a peptide sequence expected to be able to bind to an HLA antigen via internet (http://bimas.dcrt.nih.gov/molbio/hla_bind/). Thus, one can prepare the variant peptide above on the basis of the motif and the like.

For example, as hereinbefore described, the motif of an antigen peptide being capable of binding to HLA-A24 and being presented is known as a sequence characterized in that, in an 8 to 11 amino acids peptide, the amino acid at position 2 is tyrosine, phenylalanine, methionine or tryptophan, and the C terminal amino acid is phenylalanine, leucine, isoleucine, tryptophan or methionine (J. Immunol., 152: p 3913, 1994; Immunogenetics, 41: p 178, 1995; J. Immunol., 155: p 4307, 1994). As for HLA-A2, the motif is known as a sequence characterized in that, in a 8 to 11 amino acids peptide, the amino acid at position 2 is leucine, methionine, valine, isoleucine or glutamine and the C terminal amino acid is valine or leucine (Immunogenetics, 41: p 178, 1995; J. Immunol., 155: p 4749, 1995). Furthermore, some peptide sequences that are expected to be able to bind to an HLA antigen are published via internet (httt://bimas.dcrt.nih.gov/molbio/hla bind/). Amino acids having similar characteristics to those available for the motif above are also acceptable. Thus, the present invention includes a variant peptide comprising an amino acid sequence which is the same as the amino acid sequence of the peptide of the present invention except that an amino acid(s) at positions available for substitution in light of the motif (in the case of HLA-A24 and HLA-A2, position 2 and C-terminus) is substituted by another amino acid, preferably by an amino acid expected to provide a binding activity from the result of internet search, etc., and having an activity of binding to the HLA antigen and being recognized by a CTL.

More preferably, the present invention includes a variant peptide whose amino acid(s) at the aforementioned position(s) is substituted by another amino acid known to be available in light of the motif and having the aforementioned activity. Thus, in the case of HLA-A24-binding peptides as shown in SEQ ID NOS: 3 to 23, examples of variant peptides include those comprising an amino acid sequence which is the same as the amino acid sequence of any one of SEQ ID NOS: 3 to 23 except that the amino acid at position 2 is substituted by tyrosine, phenylalanine, methionine or tryptophan, and/or the C terminal amino acid by phenylalanine, leucine, isoleucine, tryptophan or methionine, and being capable of binding to HLA-A24 antigen and being recognized by a CTL. Above all, a peptide whose amino acid at position 2 is substituted by tyrosine is more preferred.

More preferably, the variant peptide consists of an amino acid sequence which is the same as the amino acid sequence of any one of SEQ ID NOS: 3 to 5 except that the amino acid at position 2 is substituted by tyrosine, phenylalanine, methionine or tryptophan, and/or the C terminal amino acid by phenylalanine, leucine, isoleucine, tryptophan or methionine, and is capable of binding to HLA-A24 antigen and is recognized by a CTL.

In the case of HLA-A2-binding peptides as shown in SEQ ID NOS: 24 to 33, a preferable variant peptide is that comprising an amino acid sequence which is the same as the amino acid sequence of any one of SEQ ID NOS: 24 to 33 except that the amino acid at position 2 is substituted by leucine, methionine, valine, isoleucine or glutamine and/or the C terminal amino acid by valine or leucine, and being capable of binding to HLA-A2 antigen and being recognized by a CTL.

The peptide of the present invention further includes an epitope peptide comprising the tumor antigen peptide of the present invention mentioned above.

Recently, a peptide composed of multiple (plural) CTL epitopes (antigen peptides) ligated together (“epitope peptide”) has been shown to induce CTLs efficiently. For example, it has been reported that a peptide (about 30-mer) composed of CTL epitopes originated from a tumor antigen protein PSA each restricted to HLA-A2-, -A3, -A11, or B53 ligated together induced in vivo CTLs specific for respective CTL epitopes (Journal of Immunology 1998, 161: 3186-3194).

In addition, a peptide (epitope peptide) composed of a CTL epitope and a helper epitope ligated together has been shown to induce a CTL efficiently. In this context, “helper epitope” means a peptide capable of activating CD4-positive T cells (Immunity., 1:751, 1994), and examples thereof include HBVc128-140 of hepatitis B virus origin, TT947-967 of tetanus toxin origin, etc. CD4⁺ T cells activated with the helper epitope exert some activities including induction and maintenance of CTLs, and activation of effectors such as macrophages, etc, and hence are considered to be important in the immunological anti-tumor response. As a specific example of the peptide composed of a helper epitope and a CTL epitope ligated together, it is reported that a DNA (minigene) composed of six kinds of HBV-derived HLA-A2-restricted antigen peptides, three kinds of HLA-A11-restricted antigen peptides and a helper epitope induced in vivo CTLs directed to the respective epitopes efficiently (Journal of Immunology 1999, 162: 3915-3925). Practically, a peptide composed of a CTL epitope (a tumor antigen peptide corresponding to position 280-288 of melanoma antigen gp100) and a helper epitope (tetanus toxin-derived T helper epitope) ligated has been subjected to clinical test (Clinical Cancer Res., 2001, 7:3012-3024).

Accordingly, the tumor antigen peptide of the present invention also includes a peptide (epitope peptide) composed of multiple epitopes including the peptide of the present invention ligated together and having an activity of inducing a CTL.

In this respect, the “epitope peptide” is defined as a peptide composed of two or more CTL epitopes (tumor antigen peptides) ligated together, or (b) a peptide composed of a CTL epitope(s) and a helper epitope(s) ligated together, which is processed in an antigen-presenting celli(s) to give a tumor antigen peptide(s) then being presented by the cell(s) and induces a CTL(s)

When a CTL epitope is ligated to the peptide of the present invention, the CTL epitope may be that derived from the amino acid sequence of AMACR as shown in SEQ ID NO: 2 and being restricted to HLA-A1, -A0201, -A0204, -A0205, -A0206, -A0207, -A11, -A24, -A31, -AG801, -B7, -B8, -B2705, -B37,-B55, -Cw0401, -Cw0602, and the like. CTL epitopes derived from other tumor antigen proteins are also usable. Plural number of CTL epitopes can be ligated together, and the length of a CTL epitope may be about 8-14 amino acids based on the analysis of antigen peptides binding to various HLA molecules (Immunogenetics, 41: 178, 1995).

When the a helper epitope is ligated to the peptide of the present invention, the helper epitope may be the aforementioned HBVc128-140 of hepatitis B virus origin, TT947-967 of tetanus toxin origin, etc. The helper epitope may be about 13-30 amino acids, preferably about 13-17 amino acids in length.

The peptide (epitope peptide) composed of multiple epitopes ligated together can be prepared by the aforementioned conventional method for peptide synthesis. It can also be prepared by a conventional method for DNA synthesis and genetic engineering on the basis of the sequence information of a polynucleotide encoding an epitope peptide composed of multiple epitopes ligated together. That is, an epitope peptide composed of multiple epitopes ligated together can be prepared by inserting a polynucleotide encoding the epitope peptide into a known expression vector, transforming a host cell with the resultant recombinant expression vector, culturing the transformant, and recovering the desired epitope peptide from the culture. These processes can be conducted according to, for example, a method described in literatures (Molecular Cloning, T. Maniatis et al., CSH Laboratory (1983), DNA Cloning, D M. Glover, IRL PRESS (1985)).

The epitope peptide produced as mentioned above, which is composed of multiple epitopes ligated together, can be examined for CTL-inducing activity in vitro by means of an assay as mentioned above, or in vivo by means of an assay described in WO02/47474 or Int J. Cancer. 100, 565-570 (2002) using a model animal for human.

Also, the amino group of the N-terminal amino acid or the carboxyl group of the C-terminal amino acid of the peptide of the present invention can be modified. The peptide undergone such modification also falls within the scope of the present invention.

The modification of the amino group of the N-terminal amino acid involves 1 to 3 groups selected from C₁₋₆ alkyl group, phenyl group, cycloalkyl group and acyl group, for example. The acyl group specifically includes C₁₋₆ alkanoyl group, C₁₋₆ alkanoyl group substituted by phenyl group, carbonyl group substituted by C₅₋₇ cycloalkyl group, C₁₋₆ alkylsulfonyl group, phenylsulfonyl group, C₂₋₆ alkoxycarbonyl group, alkoxycarbonyl group substituted by phenyl group, carbonyl group substituted by C₅₋₇ cycloalkoxy group, phenoxycarbonyl group, etc.

The peptide modified at the carboxyl group of the C-terminal amino acid may be ester or amide form. The ester specifically includes C₁₋₆ alkyl ester, C₀₋₆ alkyl ester substituted by phenyl group, C₅₋₇ cycloalkyl ester, etc. The amide specifically includes amide, amide substituted by one or two C₁₋₆ alkyl groups, amide substituted by one or two C₀₋₆ alkyl groups that are substituted by phenyl group, amide forming 5- to 7-membered azacycloalkane inclusive of nitrogen atom of amide group, etc.

### 3) Nucleic Acid of the Present Invention

The nucleic acid of the present invention specifically refers to
(1) a nucleic acid comprising the polynucleotide encoding AMACR, and
(2) a nucleic acid comprising the polynucleotide encoding the peptide of the present invention.

### (1) Polynucleotide Encoding AMACR and Nucleic Acid Comprising the Same

The polynucleotide encoding AMACR can be cDNA or mRNA, cRNA or genomic DNA of various cells or tissues such as those originated from prostate cancer, or synthetic DNA. It may be in either form of single or double strands. Specifically, the polynucleotide includes the followings:
(a) a polynucleotide comprising the base sequence of SEQ ID NO: 1;
(b) a polynucleotide comprising a base sequence encoding the amino acid sequence of SEQ ID NO: 2; and
   a polynucleotide comprising a base sequence similar to that of the polynucleotide (a) or (b).

In this respect, the base sequence of SEQ ID NO: 1 corresponds to the open reading frame of human AMACR gene which is registered with GenBank database under Accession No. NM_{—}014324. The amino acid sequence of SEQ ID NO: 2 corresponds to that of human AMACR which is registered with GenBank database under Accession No. NM_{—}014324, Accession No. NP_{—}055139.

The aforementioned (a) polynucleotide comprising the base sequence of SEQ ID NO: 1 and (b) polynucleotide comprising a base sequence encoding the amino acid sequence of SEQ ID NO: 2 specifically include a polynucleotide consisting of the base sequence of SEQ ID NO: 1 and a polynucleotide consisting of a base sequence encoding the amino acid sequence of SEQ ID NO: 2. Further example includes a polynucleotide consisting of a base sequence which contains the base sequence of SEQ ID NO: 1 or that encoding the amino acid sequence of SEQ ID NO: 2, to which an additional base sequence is added at the 5′- and/or 3′-terminus. “Additional base sequence” may be a base sequence encoding a structural gene other than AMACR.

Such a polynucleotide encoding AMACR is characterized in that the protein encoded by the polynucleotide has an activity as a tumor antigen protein. The activity and method of determining the same are described in “1) The Protein of the Present Invention”.

A polynucleotide comprising the base sequence of SEQ ID NO: 1 can be cloned by screening a cDNA library derived from, for example, a prostate cancer cell line such as DU 145 (ATCC Number: HTB-81) using an appropriate portion of the base sequence disclosed in GenBank Accession No. NM_{—}014324 or herein disclosed in SEQ ID NO: 1 as a probe for hybridization or a primer for PCR. One ordinary skilled in the art can easily conduct the cloning according to the method described in Molecular Cloning 2nd Edt. Cold Spring Harbor Laboratory Press (1989), etc.

A polynucleotide comprising a base sequence similar to that of the polynucleotide (a) or (b) above specifically includes the followings:

(c) a polynucleotide capable of hybridizing to a complementary strand of the polynucleotide (a) or (b) under stringent conditions, which encodes a protein having an activity as a tumor antigen protein;

(d) a polynucleotide comprising a base sequence having at least 70% sequence identity with the polynucleotide (a) or (b), which encodes a protein having an activity as a tumor antigen protein; and

(e) a polynucleotide encoding a protein comprising an amino acid sequence which is the same as the amino acid sequence encoded by the polynucleotide (a) or (b) except that one or more amino acids are deleted, substituted and/or added, wherein the protein has an activity as a tumor antigen protein.

Preferred examples include a polynucleotide consisting of a base sequence similar to that of the polynucleotide (a) or (b) above. The polynucleotide consisting of a base sequence similar to that of the polynucleotide (a) or (b) above includes the polynucleotides (c′) to (e′) below:

(c′) a polynucleotide capable of hybridizing to a complementary strand of the polynucleotide (a) or (b) under stringent conditions, which encodes a protein having an activity as a tumor antigen protein;

(d′) a polynucleotide consisting of a base sequence having at least 70% sequence identity with the polynucleotide (a) or (b), which encodes a protein having an activity as a tumor antigen protein; and

(e′) a polynucleotide encoding a protein consisting of an amino acid sequence which is the same as the amino acid sequence encoded by the polynucleotide (a) or (b) except that one or more amino acids are deleted, substituted and/or added, wherein the protein has an activity as a tumor antigen protein.

Examples of “polynucleotide capable of hybridizing to a complementary strand of the polynucleotide (a) or (b) above under stringent conditions” include a polynucleotide comprising a base sequence having at least about 40%, preferably about 60%, more preferably about 70%, still more preferably about 80%, further more preferably about 90%, and most preferably about 95% sequence identity with the base sequence of the polynucleotide (a) or (b) above, and specifically, a polynucleotide consisting of a partial sequence of the polynucleotide (a) or (b) above.

Hybridization can be conducted according to a method known per se or a method equivalent thereto, for example, a method described in a fundamental text “Molecular Cloning 2nd Edt. Cold Spring Harbor Laboratory Press (1989)”, and the like. Also, it can be performed using a commercially available library according to the instructions attached thereto.

The “stringent conditions” herein used can be determined on the basis of the melting temperature (Tm) of nucleic acids forming a complex or a nucleic acid binding to a probe as described in literatures (Berger and Kimmel, 1987, “Guide to Molecular Cloning Techniques Methods in Enzymology”, Vol. 152, Academic Press, San Diego Calif.; or “Molecular Cloning” 2nd Edt. Cold Spring Harbor Laboratory Press (1989)).

For example, hybridization can be carried out in a solution containing 6×SSC (20×SSC corresponds to 333 mm sodium citrate, 333 mM NaCl), 0.5% SDS and 50% formamide at 42° C., or in a solution containing 6×SSC (without 50% formamide) at 65° C.

Washing after the hybridization can be conducted under a condition around “1×SSC, 0.1% SDS, 37° C.”. The complementary strand preferably remains to bind to the target sense strand when washed under such washing conditions. More stringent hybridization conditions may involve washing under the conditions of around “0.5×SSC, 0.1% SDS, 42° C.” and still more stringent hybridization conditions around “0.1×SSC, 0.1% SDS, 65° C.”, although it is not limited thereto.

“Polynucleotide comprising a base sequence having at least 70% sequence identity with the polynucleotide of (a) or (b) above” includes a polynucleotide comprising a base sequence having at least about 70%, preferably about 80%, more preferably about 90%, and most preferably about 95% sequence identity with the base sequence of the polynucleotide of (a) or (b) above, and specifically, a polynucleotide consisting of a partial sequence of the polynucleotide of (a) or (b) above.

The term “sequence identity” herein used refers to identity or homology between two polynucleotides. The “sequence identity” is determined by comparing two sequences by aligning them optimally over the region corresponding to the sequence to be compared. In this context. The optimum alignment of the two polynucleotides to be compared may have an addition or deletion (e.g., “gap”). Such sequence identity can be calculated by preparing alignment using, for example, Vector NTI, ClustalW algorithm (Nucleic Acid Res., 22 (22): 4673-4680(1994)). The sequence identity can be determined using software for sequence analysis, specifically, Vector NTI or GENETYX-MAC, or a sequencing tool provided by a public database. Such a public database is commonly available at Web site (http://www.ddbj.nig.ac.ip).

A polynucleotide having such sequence identity can be prepared according to the aforementioned hybridization method, or conventional PCR reaction or a reaction for modifying a polynucleotide (deletion, addition or substitution) hereinafter described.

“Polynucleotide encoding a protein comprising an amino acid sequence which is the same as the amino acid sequence of the protein encoded by the polynucleotide (a) or (b) above except that one or more amino acids are deleted, substituted and/or added” includes a nucleic acid encoding a variant protein produced artificially or an allele variant present in a living body.

In this respect, there is no limitation regarding the number or position of amino acid modification (mutation) as far as the activity of the protein of the present invention is maintained. Criteria based on which one can determine the number or position of the amino acid residue to be deleted, substituted and/or added without reducing the activity can be obtained using a computer program well known in the art, such as DNA Star software. For example, the number of mutation would typically be within 10%, preferably 5% of the total amino acid residues. Furthermore, the amino acid introduced by substitution preferably has similar characteristics such as polarity, charge, solubility, hydrophobicity, hydrophilicity, amphipathicity, etc., to that to be removed in view of retention of structure. For instance, Ala, Val, Leu, Ile, Pro, Met, Phe and Trp are classified into nonpolar amino acids; Gly, Ser, Thr, Cys, Tyr, Asn and Gln into non-charged amino acids; Asp and Glu into acidic amino acids; and Lys, Arg and His into basic amino acids. One of ordinary skill in the art can select an appropriate amino acid(s) within the same group on the basis of these criteria.

The polynucleotide encoding such a variant protein may be prepared by various methods such as site-directed mutagenesis and PCR technique described in Molecular Cloning 2nd Edt., Cold Spring Harbor Laboratory Press (1989). It also can be prepared by a known method such as Gapped duplex or Kunkel method using a commercially available kit.

The polynucleotide encoding AMACR of the present invention as mentioned above encodes a protein having an activity as a tumor antigen protein. “Having an activity as a tumor antigen protein” means that the protein is positive in a conventional assay for the activity of a tumor antigen protein. Specifically, it refers to the characteristics that a cell expressing the polynucleotide encoding AMACR is recognized by a CTL, that is, the cell exhibits reactivity to a CTL, in other words, the protein of the present invention AMACR or a tumor antigen peptide derived therefrom activates or induces a CTL. The activity and the method of determination thereof are as described in “1) Proteins of the present invention AMACR” above.

The nucleic acid comprising the polynucleotide of the present invention may be in either form of single or double strands and may be either DNA or RNA. When the polynucleotide of the present invention is double stranded, an expression vector for expressing the protein of the present invention can be constructed by incorporating the above-mentioned polynucleotide into an expression vector. Thus, the nucleic acid of the present invention encompasses a recombinant expression vector constructed by inserting a double strand polynucleotide of the present invention to an expression vector.

A suitable expression vector can be selected depending on the host to be used, purposes, and the like, and includes plasmids, phage vectors, virus vectors, etc.

When the host is Escherichia coli, the vector may be a plasmid vector such as pUC118, pUC119, pBR322, pCR3, etc.; and a phage vector such as λZAPII, λgt11, etc. When the host is yeast, the vector may be pYES2, pYEUra3, etc. When the host is an insect cell, the vector may be pAcSGHisNT-A, etc. When the host is an animal cell, the vector may be a plasmid vector such as pCEP4, pKCR, pCDM8, pGL2, pcDNA3.1, pRc/RSV, pRc/CMV, etc; and a virus vector such as retrovirus vector, adenovirus vector, adeno-associated virus vector, etc.

The expression vector may optionally contain a factor(s) such as promoter capable of inducing expression, a gene encoding a signal sequence, a marker gene for selection, terminator, etc.

Furthermore, the expression vector may contain an additional sequence for expressing the protein as a fusion protein with thioredoxin, His-tag, GST (glutathione S-transferase), or the like, so as to facilitate isolation and purification of the protein. The vector usable in such a case includes a GST fusion protein vector containing an appropriate promoter (lac, tac, trc, trp, CMV, SV40 early promoter, etc) that functions in a host cell, such as pGEX4T; a vector containing Tag sequence (Myc, His, etc) such as pcDNA3.1/Myc-His; and a vector capable of expressing a fusion protein with thioredoxin and His such as pET32a.

By transforming a host cell with the expression vector obtained in the above, a transformed cell containing the vector of the present invention can be prepared.

The host cell usable herein includes Escherichia coli, yeast, insect cells and animal cells. Examples of Escherichia coli include strains of E. coli K-12 such as HB101, C600, JM109, DH5α and AD494 (DE3). Examples of yeast include Saccharomyces cerevisiae. Examples of animal cells include L929, BALB/c3T3, C127, CHO, COS, Vero, Hela and 293-EBNA cells. Examples of insect cells include sf9.

Introduction of an expression vector into a host cell can be done using a conventional method suited for the respective host cell above. Specifically, it can be done with calcium phosphate method, DEAS-dextran method, electroporation method, or a method using lipid for gene transfer (Lipofectamine, Lipofectin; Gibco-BRL). Following the introduction, the cell is cultured in a conventional medium containing a selection marker, whereby the transformant containing the expression vector can be selected.

The protein of the present invention (AMACR) can be produced by culturing the transformant under appropriate conditions. The resultant protein may be further isolated and purified according to standard biochemical procedures. The purification procedure includes salting out, ion exchange chromatography, absorption chromatography, affinity chromatography, gel filtration chromatography, etc. When the protein of the present invention is expressed as a fusion protein with thioredoxin, His tag, GST, or the like, as mentioned above, it can be isolated and purified by an appropriate purification procedure making use of the characteristics of such a fusion protein or tag.

### (2) Polynucleotide Encoding the Peptide of the Present Invention and Nucleic Acid Comprising the Same

As mentioned above, a nucleic acid comprising a polynucleotide encoding the peptide of the present invention falls within the scope of the nucleic acid of the present invention.

The polynucleotide encoding the peptide of the present invention may be in either form of DNA or RNA and single- or double-strand. The polynucleotide can be easily prepared on the basis of information about the amino acid sequence of the peptide or DNA encoding the same. Specifically, it can be prepared by a conventional method such as DNA synthesis or amplification by PCR.

Specifically, the polynucleotide encoding the peptide of the present invention includes a polynucleotide encoding the epitope peptide as mentioned above.

The nucleic acid comprising the polynucleotide encoding the peptide of the present invention may be in either form of single- or double-strand and DNA or RNA. When the polynucleotide of the present invention is double-stranded, a recombinant expression vector for expressing the peptide (epitope peptide) of the present invention can be constructed by introducing the above-mentioned polynucleotide into an expression vector.

The expression vector, host cell, method for transforming a host cell, and the like herein used are similar to those described in (1) above.

4) Antigen-Presenting Cell of the Present Invention

Examples below demonstrate that stimulation with the peptide of the present invention induced a CTL, that is, that an antigen presenting cell (dendritic cell) presenting a complex between the peptide of the present invention and an HLA antigen induced a CTL specifically recognizing that cell. Therefore, an antigen-presenting cell can be prepared by bringing a cell having an antigen-presenting ability into contact in vitro with any one of the protein, peptide and nucleic acid of the present invention as mentioned above. Specifically, the present invention provides a method of preparing an antigen-presenting cell characterized in that a cell having an antigen-presenting ability isolated from a tumor patient is brought into contact in vitro with any one of the protein, peptide and nucleic acid of the present invention and the antigen presenting cell prepared by the method.

In this context, the “cell having an antigen-presenting ability” is not limited to a particular cell and may be any cell that expresses on the surface an HLA antigen capable of presenting the peptide of the present invention; however, dendritic cells known to have especially high antigen-presenting ability are preferred.

Further, any of the protein, peptide and nucleic acid of the present invention may be used for preparing the antigen-presenting cell of the present invention from a cell having an antigen-presenting ability.

The antigen-presenting cell of the present invention can be prepared by isolating, from a tumor patient, cells having an antigen-presenting ability, pulsing the cells in vitro with the protein or peptide of the present invention, and allowing the cells to present a complex between an HLA antigen and the peptide of the present invention (Cancer Immunol. Immunother., 46: 82, 1998; J. Immunol. 158: p 1796, 1997; Cancer Res., 59:1184, 1999). When dendritic cells are used, the antigen-presenting cell of the present invention may be prepared, for example, by isolating lymphocytes from peripheral blood of a tumor patient using Ficoll method, removing non-adherent cells, incubating the adherent cells in the presence of GM-CSF and IL-4 to induce dendritic cells, and incubating and pulsing said dendritic cells with the protein or peptide of the present invention.

When the antigen-presenting cell of the present invention is prepared by introducing the nucleic acid of the present invention into the cell having an antigen-presenting ability, the nucleic acid may be in the form of DNA or RNA. In particular, DNA may be used according to the teaching in Cancer Res., 56:5672, 1996 or J. Immunol., 161: p 5607, 1998, and RNA according to the teaching in J. Exp. Med., 184:p 465, 1996, for example.

The antigen presenting cell of the present invention is characterized in that the cell presents a complex between the peptide of the present invention and an HLA antigen, and specifically includes an antigen presenting cell which is a dendritic cell and presents a complex between the peptide consisting of the amino acid sequence of SEQ ID NO: 3, 4, or 5 and HLA-A24 antigen on the cell surface. Such an antigen presenting cell can be prepared by isolating cells having an antigen-presenting ability from a HLA-A24⁺ prostate cancer patient, pulsing the cells in vitro with the peptide consisting of the amino acid sequence of SEQ ID NO: 3, 4, or 5, and allowing the cells to present a complex between the peptide and HLA-A24 antigen.

### 5) CTL of the Present Invention

The following Examples demonstrate that stimulation with the peptide of the present invention induced a CTL, that is, that an antigen presenting cell (dendritic cell) presenting a complex between the peptide of the present invention and an HLA antigen induced a CTL specifically recognizing the cell. Thus, any one of the protein, peptide and nucleic acid of the present invention can be used to induce a CTL in vitro by being brought into contact with peripheral blood lymphocytes. Specifically, the present invention provides a method of inducing a CTL wherein peripheral blood lymphocytes from a tumor patient are brought into contact in vitro with any one of the protein, peptide and nucleic acid of the present invention and the CTL induced thereby.

For melanoma, adoptive immunotherapy has shown a therapeutic effect, wherein tumor-infiltrating T cells obtained from a patient were cultured ex vivo in large quantities and returned into the same patient (J. Natl. Cancer. Inst., 86: 1159, 1994). Further, in mouse melanoma, suppression of metastasis has been observed by stimulating splenocytes with a tumor antigen peptide TRP-2 in vitro to induce proliferation of a CTL specific to the tumor antigen peptide, and administering the CTL to a melanoma-grafted mouse (J. Exp. Med., 185:453, 1997). This resulted from the in vitro proliferation a CTL that specifically recognizes the complex between an HLA antigen on antigen-presenting cells and the tumor antigen peptide. Accordingly, a therapeutic method comprising stimulating in vitro peripheral blood lymphocytes from a patient with the protein, peptide or nucleic acid of the present invention to proliferate a tumor-specific CTL, and returning the CTL into the patient is believed to be effective.

The CTL used in the adoptive immunotherapy can be prepared by isolating peripheral blood lymphocytes from a patient and stimulating the lymphocytes in vitro with the protein, peptide or nucleic acid of the present invention (Journal of Experimental Medicine 1999, 190:1669).

The CTL of the invention is characterized in that it is induced by bringing peripheral blood lymphocytes into contact in vitro with any one of the protein, peptide and nucleic acid of the present invention, and may be either a single CTL clone or CTL mixture (group) composed of various CTL clones. A specific example of such a CTL is that specifically recognizing a complex between the peptide consisting of the amino acid sequence of SEQ ID NO: 3, 4, or 5 and HLA-A24 antigen.

### (6) Pharmaceutical Composition of the Present Invention

The protein, peptide and nucleic acid of the present invention as mentioned above can be an active ingredient of a pharmaceutical composition in an appropriate form for each substance. The pharmaceutical composition of the present invention can be an active ingredient of an inducer of CTL, that is, a cancer vaccine, which is described in detail below.

### (1) Inducer of CTL Comprising the Protein of the Invention as an Active Ingredient

The protein of the present invention AMACR has an activity of inducing CTLs and therefore, the protein of the present invention can be used as an active ingredient of a medicament for the treatment or prevention of tumor (cancer vaccine). The inducer of CTL comprising the protein of the present invention as an active ingredient exerts a therapeutic or preventive effect on tumor. The protein, when administered to a tumor patient, is incorporated by antigen-presenting cells and intracellularly degradated; the resultant tumor antigen peptide(s) generated by the intracellular degradation binds to an HLA antigen to form a complex; the complex is then presented on the surface of antigen-presenting cells; and a CTL specific for the complex efficiently proliferates in the body and destroys the tumor cells. In this way, treatment or prevention of tumor is achieved.

The inducer of CTL comprising the protein of the present invention as an active ingredient can be administered to any tumor patient who is positive for AMACR protein. Specifically, it can be used for the prevention or treatment of, for example, prostate cancer, or cancer (tumor) such as bowel cancer, ovarian cancer, bladder cancer, lung cancer, renal cell cancer, lymphoma, melanoma, liver cancer, gastric cancer, pancreas cancer or uterine cancer.

The inducer of CTL comprising the protein of the present invention as an active ingredient may be administered as a mixture with, or together with, a pharmaceutically acceptable carrier, for example, an appropriate adjuvant, so that cellular immunity can be established effectively.

Examples of adjuvant applicable include those described in a literature (Clin. Microbiol. Rev., 7:277-289, 1994). Specifically, the followings are contemplated: a component derived from a microorganism or derivatives thereof, cytokines, a component derived from a plant or derivatives thereof, a component derived from a marine organism or derivatives thereof, mineral gels such as aluminium hydroxide, surfactants such as lysolecithin and Pluronic® polyols, polyanion, peptide, oil emulsion (emulsion preparation) and the like. In addition, liposomal preparations, particulate preparations in which the ingredient is bound to beads having a diameter of several μm, preparations in which the ingredient is attached to lipids, microsphere preparations, and microcapsules are also contemplated.

In this context, the “component derived from a microorganism or derivatives thereof” can be specifically classified into (1) killed bacteria, (2) Cell Wall Skeleton (hereinafter, “CWS”) derived from bacteria, and (3) a particular component derived from a microorganism and derivatives thereof.

(1) Examples of the killed bacteria include powdery hemolytic streptococcus (e.g., Picibanil®, Chugai Co., Ltd.), cocktail of killed bacterium suspension (e.g., Broncasma Berna®, Sanwa Kagaku Kenkyusho Co., Ltd) or killed bacteria of Mycobacterium tuberculosis, and the like.

(2) Examples of CWS derived from bacteria include CWS from Microbacterium (e.g., Mycobacterium bovis CWS), CWS from Nocardia (e.g., Nocardia rubra CWS), Corynebacterium CWS, etc.

(3) Examples of a particular component derived from a microorganism and derivatives thereof include microorganism-derived polysaccharides such as polysaccharides from Mycobacterium tuberculosis (e.g., Ancer®, Zeria Pharmaceutical Co., Ltd.); polysaccharides from Basidiomycetes (Lentinan®, Ajinomoto, Co., Ltd.,; Krestin®, Sankyo, Co., Ltd.; Basidiomycetes, Coriolus versicolor (Fr) Quel); muramyl dipeptide (MDP) associated compounds; lipopolysaccharides (LPS); lipid A (MPL) associated compounds; glycolipids trehalose dimycolate (TDM); bacterium DNA (e.g., CpG oligonucleotide); and derivatives thereof.

These microorganism-derived components and derivatives thereof can be available from commercial source or can be produced and isolated according to the methods described in known literatures (e.g., Cancer Res., 33, 2187-2195 (1973); J. Natl. Cancer Inst., 48, 831-835(1972), J. Bacteriol., 94, 1736-1745 (1967); Gann, 69, 619-626 (1978), J. Bacteriol., 92, 869-879 (1966) or J. Natl. Cancer Inst., 52, 95-101 (1974)).

The term “cytokine”, for example, refers to IFN-α, IL-12, GM-CSF, IL-2, IFN-γ, IL-18 or IL-15. The cytokine may be a product of nature or genetic engineering. When the cytokine is commercially available, one can pursue and use the same. Alternatively, cytokine can be prepared recombinantly by cloning a desired gene in a conventional manner on the basis of the base sequence registered with database such as GenBank, EMBL or DDBJ, ligating the gene into an appropriate expression vector, transforming a host cell with the resultant recombinant expression vector, and allowing the cell to express and produce the intended cytokine.

Examples of the “component derived from a plant or derivatives thereof” include saponin-derived component Quil A (Accurate Chemical & Scientific Corp), QS-21 (Aquila Biopharmaceuticals Inc.), or glycyrrhizin (SIGMA-ALDRICH, etc.).

Examples of the “component derived from a marine organism or derivatives thereof” include sponge-derived glycolipid α-galactosylceramide.

Examples of oil emulsion (emulsion preparation) include emulsion preparations of water-in-oil type (w/o), oil-in-water type (o/w) and water-in-oil-in-water type (w/o/w).

In the water-in-oil type (w/o) emulsion preparation, an active ingredient is dispersed in water used as the disperse phase. In the oil-in-water type (o/w) emulsion preparation, an active ingredient is dispersed in water used as the disperse medium. Further, in the water-in-oil-in-water type (w/o/w) emulsion preparation, an active ingredient is dispersed in water which is the most internal phase. Such emulsion preparations can be produced in accordance with the teaching in, for example, JP-A-8-985, JP-A-9-122476, or the like.

The “liposomal preparation” refers to a microparticle wherein an active ingredient is encapsulated in a liposome having a lipid bilayer structure in the water phase or within the lipid bilayer. Representative lipids for preparation of the liposome include phosphatidyl choline, sphingomyelin, etc. Dicetyl phosphate, phosphatidic acid, phosphatidyl serine or the like that confers charge may also be added for stabilization of liposomes. The method of producing liposomes include ultrasonic method, ethanol injection method, ether injection method, reverse phase evaporation method, French press extraction method, and the like.

The “microsphere preparation” refers to a microparticle composed of a homogeneous polymer matrix wherein an active ingredient is dispersed in the matrix. The matrix can be composed of a biodegradable polymer such as albumin, gelatin, chitin, chitosan, starch, polylactic acid, polyalkyl cyanoacrylate, and the like. The microsphere preparation can be prepared by any of known methods without limitation, including those described in literatures (Eur. J. Pharm. Biopharm. 50:129-146, 2000; Dev. Biol. Stand. 92:63-78, 1998; Pharm. Biotechnol. 10:1-43, 1997, etc.).

The “microcapsule preparation” refers to a microparticle containing an active ingredient as a core substance which is enveloped with a film. The coating material used for the film includes a film-forming polymer such as carboxymethylcellulose, cellulose acetate phthalate, ethyl cellulose, gelatin, gelatin/acacia, nitrocellulose, polyvinyl alcohol, hydroxypropyl cellulose, and the like. The microcapsule preparation can be prepared by coacervation method, surface polymerization, and the like.

Administration may be achieved, for example, intradermally, subcutaneously, intramuscularly, or intravenously. Although the dosage of the protein of the present invention in the formulation to be administered may be adjusted as appropriate depending on, for example, the disease to be treated, the age and the body weight of the patient, it is usually within the range of 0.0001 - 1000 mg, preferably, 0.001-100 mg, more preferably, 0.01-10 mg, which can be administered once in every several days to every several months.

### (2) Inducer of CTL Comprising a Peptide of the Present Invention as an Active Ingredient

The peptide of the present invention has an activity of inducing a CTL. The induced CTL can exert an anti-tumor effect through cytotoxic action or production of lymphokines. Accordingly, the peptide of the present invention can be used as an active ingredient of a medicament for the treatment or prevention of tumor (cancer vaccine). When an inducer of CTL comprising the peptide of the present invention as an active ingredient is administered to a tumor patient, the peptide of the present invention is presented to an HLA antigen in antigen-presenting cells. Then, a CTL specific for the presented binding complex between the HLA antigen and the peptide of the present invention proliferates, which in turn destroys tumor cells. In this way, the treatment or prevention of tumor in a patient can be achieved.

The inducer of CTL comprising the peptide of the present invention as an active ingredient can be administered to any tumor patient who is positive for AMACR protein. Specifically, it can be used for the prevention or treatment of, for example, prostate cancer, or cancer (tumor) such as bowel cancer, ovarian cancer, bladder cancer, lung cancer, renal cell cancer, lymphoma, melanoma, liver cancer, gastric cancer, pancreas cancer or uterine cancer.

The inducer of the present invention may comprise as an active ingredient a single CTL epitope (peptide of the present invention) or a epitope peptide composed of the peptide of the present invention and other peptide(s) (CTL epitope or helper epitope) ligated together. Recently, an epitope peptide composed of multiple (plural) CTL epitopes (antigen peptides) ligated together has been shown to have an activity of inducing CTLs efficiently. For example, it has been reported that an epitope peptide of approximately 30-mer composed of CTL epitopes each restricted to HLA-A2-, -A3,- -A11 or B53 originated from tumor antigen protein PSA ligated induced CTLs specific for respective CTL epitopes (Journal of Immunology 1998, 161: 3186-3194). In addition, it has been reported that an epitope peptide composed of a CTL epitope and a helper epitope ligated can induce a CTL efficiently. When the peptide of the present invention is administered in the form of an epitope peptide, the peptide is incorporated into antigen-presenting cells; then the antigen peptide generated by intracellular degradation binds to an HELA antigen to form a complex; the complex is presented on the surface of antigen-presenting cells in high density; a CTL specific for the complex efficiently proliferates in the body, and destroys tumor cells. In this way, treatment or prevention of tumor is achieved.

Specific examples of the inducer of the present invention include one comprising as an active ingredient the tumor antigen peptide consisting of the amino acid sequence of any one of SEQ ID NOS: 3 to 33, preferably the amino acid sequence of SEQ ID NO: 3, 4 or 5.

The inducer of CTL comprising a peptide of the present invention as an active ingredient may be administered in a mixture with, or together with, a pharmaceutically acceptable carrier, for example, an appropriate adjuvant, so that cellular immunity can be established effectively.

Examples of adjuvant applicable include those described in a literature (Clin. Microbiol. Rev., 7:277-289, 1994). Specifically, the followings are contemplated: a component derived from a microorganism or derivatives thereof, cytokines, a component derived from a plant or derivatives thereof, a component derived from a marine organism or derivatives thereof, mineral gels such as aluminium hydroxide, surfactants such as lysolecithin and Pluronic® polyols, polyanion, peptides, oil emulsion (emulsion preparation) and the like. In addition, liposomal preparations, particulate preparations in which the ingredient is bound to beads having a diameter of several μm, preparations in which the ingredient is attached to lipids, microsphere preparations, and microcapsules are also contemplated. Concrete examples of these adjuvants are the same as those described in the above “6)-1) Inducer of CTL comprising the protein of the present invention as an active ingredient”.

Administration may be achieved, for example, intradermally, subcutaneously, intramuscularly, or intravenously. Although the dosage of the peptide of the present invention in the formulation to be administered may be adjusted as appropriate depending on, for example, the disease to be treated, the age and the body weight of the patient, it is usually within the range of 0.0001-1000 mg, preferably 0.001-1000 mg, more preferably 0.1-10 mg, which can be administered once in every several days to every several months.

### (3) Inducer of CTL Comprising the Nucleic Acid of the Present Invention as an Active Ingredient

The nucleic acid of the present invention has an activity of including a CTL and thus can be an active ingredient of a medicament for the treatment or prevention of tumor (cancer vaccine). The inducer of CTL comprising the nucleic acid of the present invention as an active ingredient, when administered, can exert a therapeutic or preventive effect on tumor through the expression of the nucleic acid.

For example, when the nucleic acid of the present invention incorporated into an expression vector is administered to a tumor patient in the following manner, the tumor antigen protein is highly expressed in antigen-presenting cells. Thereafter, tumor antigen peptides generated by intracellular degradation form a complex with an HLA antigen; the complex is then presented on the surface of antigen-presenting cells in high density; and tumor-specific CTLs proliferate in the body efficiently and destroy tumor cells. In this way, treatment or prevention of tumor is achieved.

The inducer of CTL comprising the nucleic acid of the present invention as an active ingredient can be administered to any tumor patient who is positive for AMACR. Specifically, it can be used for the prevention or treatment of, for example, prostate cancer, or cancer (tumor) such as bowel cancer, ovarian cancer, bladder cancer, lung cancer, renal cell cancer, lymphoma, melanoma, liver cancer, gastric cancer, pancreas cancer or uterine cancer.

Administration and introduction of the nucleic acid of the present invention into cells may be achieved by using a viral vector or by other procedures (Nikkei-Science, April, 1994, pp. 20-45; Gekkan-Yakuji, 36(1), 23-48 (1994); Jikken-Igaku-Zokan, 12(15), 1994, and references cited therein).

The method of introducing a viral vector may comprises incorporation of the DNA of the present invention into a DNA or RNA virus such as retrovirus, adenovirus, adeno-associated virus, herpesvirus, vaccinia virus, poxvirus, poliovirus, or Sindbis virus, and introduction of the virus into cells. Above all, the method involving a retrovirus, adenovirus, adeno-associated virus, or vaccinia virus is particularly preferred.

Other than the above method, a method wherein an expression plasmid is directly injected intramuscularly (DNA vaccination), liposome method, Lipofectin method, microinjection, calcium phosphate method and electroporation are exemplified, and among them DNA vaccination and liposome method are particularly preferred.

The nucleic acid of the present invention can act as a medicament in practice in, for example, an in vivo method wherein the nucleic acid is directly introduced into the body, or an ex vivo method wherein the nucleic acid is introduced extracorporeally into a certain cell obtained from a human subject and the cell is reintroduced into the body of the subject (Nikkei-Science, April, 1994, pp. 20-45; Gekkan-Yakuji, 36(1), 23-48 (1994); Jikkenn-Igaku-Zokan, 12(15), 1994; and references cited therein). An in vivo method is more preferred.

In case of the in vivo method, administration can be effected through any appropriate route depending on the disease and symptom to be treated and other factors. For example, it may be administered via intravenous, intraarterial, subcutaneous, intracutaneous, intramuscular route, or the like. When administered in the in vivo method, the nucleic acid of the present invention may be formulated into a liquid preparation, and typically into an injectable form containing the nucleic acid of the present invention as an active ingredient, to which a conventional carrier may also be added, if necessary. As to a liposome or membrane-fused liposome (such as Sendai virus (HVJ)-liposomes) containing the nucleic acid of the present invention, a liposomal formulation in the form of suspension, frozen preparation, centrifugally-concentrated frozen preparation, or the like may be accepted.

Although the dosage of the nucleic acid of the present invention in the formulation to be administered may be adjusted as appropriate depending on, for example, the disease to be treated, the age and the body weight of the patient, it is usually, as the amount of polynucleotide in the nucleic acid, within the range of 0.0001-100 mg, preferably, 0.001-10 mg, which can be administered once in every several days to every several months.

Recently, a polynucleotide encoding an epitope peptide composed of multiple (plural) CTL epitopes (antigen peptides) ligated or of a CTL epitope(s) and a helper epitope(s) ligated has been shown to induce CTLs in vivo efficiently. For example, it is reported that a DNA (minigene) encoding an epitope peptide composed of six kinds of HBV-originated HLA-A2-restricted antigen peptides, three kinds of HLA-A11-restricted antigen peptides and a helper epitope ligated induced in vivo CTLs directed to the respective epitopes efficiently (Journal of Immunology 1999, 162: 3915-3925).

Accordingly, a polynucleotide prepared by ligating one or more polynucleotides each encoding the peptide of the present invention, and optionally other polynucleotide(s) encoding different peptide(s), can be an active ingredient of an inducer of CTL when introduced into an appropriate expression vector. Such an inducer of CTL may be applied in the same administration manner or form that described above.

### (4) Inducer of CTL Comprising the Antigen Presenting Cell of the Present Invention as an Active Ingredient

The antigen presenting cell of the present invention has an activity of inducing a CTL and thus can be an active ingredient of a medicament for the treatment or prevention of tumor (cancer vaccine). The inducer of CTL comprising the antigen presenting cell of the present invention as an active ingredient can exert a therapeutic or preventive effect on tumor through administration of the antigen presenting cell to a tumor patient.

The inducer of CTL comprising the antigen presenting cell of the present invention as an active ingredient can be administered to any tumor patient who is positive for AMACR. Specifically, it can be used for the prevention or treatment of, for example, prostate cancer, or cancer (tumor) such as bowel cancer, ovarian cancer, bladder cancer, lung cancer, renal cell cancer, lymphoma, melanoma, liver cancer, gastric cancer, pancreas cancer or uterine cancer.

The inducer of CTL comprising the antigen presenting cell as an active ingredient preferably contains physiological saline, phosphate buffered saline (PBS), medium, or the like to stably maintain the antigen-presenting cell. It may be administered, for example, intravenously, subcutaneously, or intradermally. Dosage of the inducer is exemplified in the previous literature. Reintroduction of the inducer of CTL comprising the antigen-presenting cell as an active ingredient into a AMACR-positive patient can cause efficient induction of a specific CTL in the body of the patient, and, result in the treatment of tumor.

### (5) Cancer Vaccine Comprising the CTL of the Present Invention as an Active Ingredient

The CTL of the present invention has a cytotoxic activity against tumor cells and thus can be an active ingredient of a medicament for the treatment or prevention of tumor (cancer vaccine).

The pharmaceutical composition comprising the CTL of the present invention as an active ingredient for treating or preventing tumor can be administered to any tumor patient who is positive for AMACR. Specifically, it can be used for the prevention or treatment of, for example, prostate cancer, or cancer (tumor) such as bowel cancer, ovarian cancer, bladder cancer, lung cancer, renal cell cancer, lymphoma, melanoma, liver cancer, gastric cancer, pancreas cancer or uterine cancer.

The pharmaceutical composition comprising the CTL of the present invention as an active ingredient for treating or preventing tumor preferably contains physiological saline, phosphate buffered saline (PBS), medium, or the like to stably maintain the CTL. It may be administered, for example, intravenously, subcutaneously, or intradermally. Reintroduction of the pharmaceutical composition comprising the CTL of the present invention as an active ingredient into a AMACR-positive patient can cause promotion of the cytotoxic activity of CTLs against tumor cells in the body of the patient, followed by destruction of the tumor cells, and result in treatment of tumor.

### 7) Antibody Against the Peptide of the Present Invention

The present invention provides an antibody capable of specifically binding to the peptide of the present invention. The antibody of the present invention is not limited in terms of the form, and may be a polyclonal or monoclonal antibody raised against the peptide of the present invention.

The antibody of the present invention may not be limited in any sense provided that it specifically binds to the peptide of the present invention as mentioned above, and the specific example is an antibody that specifically binds to a tumor antigen peptide consisting of the amino acid sequence of any one of SEQ ID NOS: 3 to 33, preferably the amino acid sequence of SEQ ID NO: 3, 4 or 5.

Methods of preparing an antibody are well known in the art and the antibody of the present invention can be prepared according to any one of these conventional methods (Current protocols in Molecular Biology edit. Ausubel et al. (1987) Publish. John Wiley and Sons. Section 11.12-11.13, Antibodies; A Laboratory Manual, Lane, H, D. et al., ed., Cold Spring Harber Laboratory Press, New York 1989).

Specifically, the antibody of the present invention can be obtained by immunizing a non-human animal such as rabbit using the peptide of the present invention (e.g., a tumor antigen peptide consisting of the amino acid sequence of any one of SEQ ID NOS: 3 to 33) as an antigen, and recovering the antibody from serum of the immunized animal in a conventional manner. When the antibody is monoclonal, it can be obtained by immunizing a non-human animal such as mouse with the peptide of the present invention (e.g., a tumor antigen peptide consisting of the amino acid sequence of any one of SEQ ID NOS: 3 to 33), subjecting the resultant splenocyte to cell fusion with a myeloma cell to prepare a hybridoma cell, and recovering the antibody from the hybridoma cell (Current protocols in Molecular Biology edit. Ausubel et al. (1987) Publish. John Wiley and Sons. Section 11.4-11.11).

The antibody against the peptide of the present invention can also be produced while enhancing the immunological response using different adjuvants depending on the host. Examples of the adjuvants include Freund adjuvant; mineral gels such as aluminium hydroxide; surfactants such as lysolecithin and Pluronic® polyol, polyanion, peptides, oil emulsion, keyhole limpet hemocyanin and dinitorophenol; human adjuvants such as BCG (Bacille de Calmette-Guerin) or Corynebacterium, etc.

As mentioned above, an antibody that recognizes the peptide of the present invention and an antibody that neutralizes the activity of the peptide may easily be prepared by immunizing an animal in a conventional manner. The antibody may be used in affinity chromatography, immunological diagnostic method, and the like. Immunological diagnostic method may be selected as appropriate from immunoblotting, radioimmunoassay (RIA), enzyme-linked immunosorbent assay (ELISA), fluorescent or luminescent assay, and the like. The immunological diagnostic method would be effective for diagnosing cancer which expresses AMACR gene of the present invention such as prostate cancer.

### 8) HLA Monomer, HLA Dimer, HLA Tetramer and HLA Pentamer of the Present Invention

The present invention also provides an HLA monomer, HLA diner, HLA tetramer or HLA pentamer comprising the tumor antigen peptide of the present invention and an HLA antigen.

In cancer immunotherapy, a significant indicator for selecting a patient highly responsive to a tumor antigen (tumor antigen peptide), monitoring the therapeutic effect, or evaluating the suitability to treatment can be obtained through examination of frequency or amount of CTL precursor cells directed to the tumor antigen (tumor antigen peptide) in a patient before initiation of treatment, or examination of frequency or amount of CTLs in a patient undergoing treatment with the tumor antigen (tumor antigen peptide). An HLA monomer, HLA dimer, HLA tetramer and HLA pentamer each comprising a tumor antigen peptide and an HLA antigen are useful as a reagent in detection of a CTL specific for the antigen (antigen peptide), specifically, in the measurement of frequency or amount of the CTL.

As used herein, the HLA tetramer refers to a tetramer prepared by biotinylating a complex composed of HLA antigen α-chain and β2-microglobulin associated with a peptide (antigen peptide) (HLA monomer), and allowing to bind to avidin for tetramerization (Science 279: 2103-2106 (1998); and Science 274: 94-96 (1996)).

The HLA monomer is a monomer that is used in the preparation of the above-mentioned HLA tetramer and is formed by biotinylating an associate of HLA antigen α-chain, β2-microglobulin and an antigen peptide.

The HLA dimer is a dimer prepared by fusing HLA antigen a-chain and Ig (immunoglobulin, for example, IgG1), and binding the resultant fusion to β2-microglobulin and an antigen peptide (Proc. Natl. Acad. Sci. USA 90: 6671-6675 (1993)). The CTL specific for the antigen peptide bound to the HLA dimer can be detected by, for example, allowing labeled anti-IgG1 antibody to bind to the IgG1.

The HLA pentamer is a recently developed technique and refers to a pentamer comprising five molecules of a complex of an HLA antigen and an antigen peptide polymerized through Coiled-Coil domain. Since the complex of an HLA antigen and an antigen peptide can be labeled with fluorescence or the like, the analysis can be carried out by flow cytometry or the like similarly to the HLA tetramer (see, http://www.proimmune.co.uk/).

The HLA-monomer, dimer, tetramer and pentamer as mentioned above are all available by custom production from a manufacture such as ProImmune or BD Biosciences. At present, HLA tetramers and the like which comprise different antigen peptides are commercially available (Medical & Biological Laboratories Co., Ltd., etc.).

Examples of the HLA monomer, dimer, tetramer and pentamer of the present invention, specifically, include a HLA monomer, dimer, tetramer and pentamer each comprising the peptide consisting of the amino acid sequence of SEQ ID NO: 3, 4, or 5 and HLA-A24 antigen. Above all, an HLA tetramer or an HLA pentamer is preferred for detection of a CTL.

The HLA monomer, HLA tetramer and HLA pentamer are preferably labeled with fluorescence so that the bound CTL can be easily sorted out or detected by a known detection measure such as flow cytometry, fluorescent microscopy, and the like. Examples include HLA monomers, tetramers and dimers labeled with phycoerythrin (PE), fluorescein isothiocyanate (FITC), peridinyl chlorophyll protein (PerCP), allophycocyanin (APC), or the like.

Among HLA antigens which may be a component of the HLA monomer, diner, tetramer and pentamer of the present invention, HLA-A24 antigen (α-chain of HLA-A24 antigen) can be cloned easily by a conventional method such as PCR on the basis of a known base sequence of HLA-A2402 disclosed in Genbank Accession No. M64740. HLA-A2 antigen (α-chain of HLA-A2 antigen) can be cloned easily by a conventional method such as PCR on the basis of a known base sequence of HLA-A0201 gene disclosed in Genbank Accession No. M84379.

The β2-microglobulin which is a component of the HLA monomer, dimer, tetramer and pentamer of the present invention is preferably originated from human. The human β2-microglobulin can be cloned easily by a conventional method such as PCR on the basis of a known base sequence of human β2-microglobulin disclosed in Genbank Accession No. AB021288.

The processes for preparing the HLA monomer, diner, tetramer and pentamer are well known from the respective literatures mentioned above; however, the preparation will be hereinafter described briefly regarding the HLA tetramer.

First, an appropriate host cell such as E. coli or a mammalian cell capable of expressing a protein is transformed with an HLA-A24 α-chain expression vector and a β2-microglobulin expression vector, and allowed to express them. E. coli (e.g., BL21) is preferably used here. The resultant HLA-A24 complex in a monomeric form and a peptide of the present invention are then mixed to form a soluble HLA-peptide complex. The C-terminal sequence of HLA-A24 α-chain of the HLA-peptide complex is biotinylated with BirA enzyme. When the biotinylated HLA-peptide complex and fluorescently labeled avidin are mixed at the molar ratio of 4:1, an HLA tetramer is formed. It is preferred to purify the resulting protein in each step above by gel filtration or the like.

The HLA monomer, dimer, tetramer and pentamer described above may be used effectively as a detecting reagent for a CTL specific for an AMACR-derived tumor antigen peptide.

The CTL-detecting reagent of the present invention can be used for the following purposes, for example.
1) To examine the frequency or amount of a CTL precursor for the tumor antigen peptide of the present invention before the initiation of treatment with the protein, peptide or nucleic acid of the present invention. In doing so, responsiveness of a patient to the tumor antigen peptide can be assessed.
2) To examine the frequency or amount of CTLs in a patient under treatment with the protein, peptide or nucleic acid of the present invention. In doing so, it becomes possible to conduct monitoring of a therapeutic effect, evaluation of suitability of treatment, and confirmation of favorable progress of treatment, and the like.

Detection of a CTL can be carried out by, specifically, isolating a biological sample containing CTLs (e.g., PBMCs) from a subject patient, bringing the HLA tetramer or the like of the present invention into contact with the biological sample, and measuring the frequency or amount of the existing CTL specific for the peptide of the present invention bound to the HLA tetramer by, for example, flow cytometry.

The present invention is specifically explained by the following examples, although the examples should not be deemed to limit the present invention in any sense.

### EXAMPLE 1

### Synthesis and Selection of Candidate Peptides

### (1) Selection of Candidate Peptides

Peptides each consisting of any one of the amino acid sequences of SEQ ID NOS: 3 to 23 were selected as candidate peptides derived from the amino acid sequence of human AMACR (SEQ ID NO: 2) which were potential to bind to an HLA-A24 molecule. Further, Peptides each consisting of any one of the amino acid sequences of SEQ ID NOS: 24 to 33 were selected as candidate peptides which were potential to bind to an HLA-A2 molecule. The sequence of each of those peptides and the position on the sequence of AMACR are listed below.

TABLE 2
AMACR A24 peptides

| Name of the | | |
|---|---|---|
| peptide | Sequence | SEQ ID NO: |
| AMACR 18-22 | PFCAMVLADF | 7 |
| AMACR 115-124 | SFCRLAGHDI | 8 |
| AMACR 125-133 | NYLALSGVL | 3 |
| AMACR 158-166 | LMCALGIIM | 9 |
| AMACR 183-191 | NMVEGTAYL | 4 |
| AMACR 193-201 | SFLWKTQKL | 10 |
| AMACR 195-203 | LWKTQKLSL | 11 |
| AMACR 203-212 | LWEAPRGQNM | 12 |
| AMACR 211-219 | NMLDGGAPF | 13 |
| AMACR 221-229 | TYRTADGEF | 14 |
| AMACR 221-230 | TYRTADGEFM | 15 |
| AMACR 239-248 | QFYELLIKGL | 16 |
| AMACR 240-248 | FYELLIKGL | 5 |
| AMACR 258-267 | QMSMDDWPEM | 17 |
| AMACR 263-271 | DWPEMKKKF | 18 |
| AMACR 266-275 | EMKKKFADVF | 19 |
| AMACR 274-283 | VFAEKTKAEW | 20 |
| AMACR 344-353 | PFIGEHTEEI | 21 |
| AMACR 358-367 | GFSREEIYQL | 22 |
| AMACR 364-372 | IYQLNSDKI | 23 |
| AMACR 364-373 | IYQLNSDKII | 6 |

TABLE 3
AMACR A2 peptides

| Name of the | | |
|---|---|---|
| peptide | Sequence | SEQ ID NO: |
| AMACR 190-198 | YLSSFLWKT | 24 |
| AMACR_23-31 | VLADFGARV | 25 |
| AMACR 183-191 | NMVEGTAYL | 26 (4) |
| AMACR 75-84 | VLLEPFRRGV | 27 |
| AMACR_194-203 | FLWKTQKLSL | 28 |
| AMACR_118-127 | RLAGHDINYL | 29 |
| AMACR 212-221 | MLDGGAPFYT | 30 |
| AMACR 22-31 | MVLADFGARV | 31 |
| AMACR_285-294 | QIFDGTDACV | 32 |
| AMACR_45-54 | RLGRGKRSLV | 33 |

### (2) Peptide Synthesis

Among the peptides mentioned above, the peptides AMACR 125-133 (SEQ ID NO: 3), AMACR 183-191 (SEQ ID NO: 4), AMACR 240-248 (SEQ ID NO: 5), and AMACR 364-373 (SEQ ID NO: 6) were synthesized by Fmoc method and used in the following examples.

### EXAMPLE 2

### Evaluation of the Binding Affinity to HLA-A*2402 of the AMACR Antigen-Derived Peptide

The binding affinities to HLA-A*2402 of the peptides synthesized in Example 1 were determined by the method as described in a literature (J. Immunol. 164:2565, 2000). A cell line RMA-S-A*2402 cell, which was obtained by introducing a chimera MHC gene composed of HLA-A*2402 and H-2Kb into a mouse lymphoma cell line RMA-S lacking MHC class I molecule, was incubated at 26° C. for 18 hours. RMA-S-A*2402 cells were washed with PBS solution, suspended in culture solution OPTI-MEM (Invitrogen) containing 3 μL/mL human β₂-microglobulin and 100 μL/mL each peptide, and incubated at 26° C. for 3 hours and at 37° C. for 3 hours. The cells were washed with PBS solution and treated with anti-HLA-A24 and anti-HLA-A2 antibodies at 4° C. for 30 minutes. Furthermore, the cells were washed with PBS solution, and treated with a PE-labeled anti-mouse IgG antibody at 4° C. for 30 minutes. The cells were washed, and suspended in 1 ml of PBS solution containing 1% formalin for fixation. The cells were measured by a device for flow cytometry, FACScan (BD Bioscience), and the binding affinity of the peptide was obtained from the mean fluorescence intensity. The binding affinities of the four peptides determined are shown in FIG. 1.

An EB virus-derived peptide (EBV) and HIV virus-derived peptide (HIV), which had been reported to bind to HLA-A*2402 (J. Immunol. 158:3325, 1997 and J. Immunol. 164:2565, 2000, respectively) and was used as a positive control, showed a strong binding activity. An ovalbumin-derived peptide (SL8), which had been reported to bind to H2-Kb (Eur J Immunol. 21:2891, 1991) and was used as a negative control, showed a weak binding activity. Among the peptides evaluated, three AMACR antigen-derived peptides AMACR 125-133 (SEQ ID NO: 3), AMACR 183-191 (SEQ ID NO: 4), and AMACR 240-248 (SEQ ID NO: 5) were demonstrated to bind to HLA-A*2402 in a highly preferable manner.

### EXAMPLE 3

### CTL Induction By the AMACR Antigen-Derived Peptide From Human Peripheral Blood Mononuclear Cells (1)

A CTL was induced from peripheral blood mononuclear cells using the peptide AMACR 240-248 (SEQ ID NO: 5) that showed a strong binding activity to HLA-A*2402 in Example 2 according to the method as described in a literature (J. Immunol. 169:1611, 2002). After obtaining informed-consent, peripheral blood was collected from an HLA-A*2402-positive patient having prostate cancer, and mononuclear cells were separated by density gradient centrifugation method and cultured in AIM-V culture solution (Invitrogen). After 24-hour-cultivation, nonadherent cells were recovered and cultured in AIM-V containing 100 U/mL IL-2. For preparation of antigen-presenting cells, adherent cells were cultured in AIM-V culture solution containing 1000 U/mL IL-4 and 1000 U/mL GM-CSF for 5 days, and, after addition of 10 μM peptide, cultured for another 1 day. To the culture were then added 10 ng/mL TNF and 1000 U/mL IFN-α, and the mixture was cultured. CD8-positive T cells were separated from the nonadherent cells by means of anti-CDS antibody-bound magnetic beads, and cultured together with the antigen-presenting cells pulsed with the above peptide. The remaining nonadhesive cells after the separation of CD8-positive T cells were cultured in AIM-V medium containing 1 μg/mL PHA and 100 U/mL IL-2 for 3 days, then in a medium lacking PEA for 4 days, and stocked as antigen presenting cells for the second and third peptide-stimulation. The CD8-positive T cells that had received peptide-stimulation were subjected to the second and third peptide-stimulation on 7 and 14 days after the first peptide-stimulation by adding the stocked antigen presenting cells having been pulsed with the above peptide for 2 hours and X-ray radiated (5000 rad). After one week from the third stimulation, cytotoxic activity of T cells was measured by ⁵¹Cr release assay.

The following cells were used as target cells: T2A24 cell, which was produced by introducing HLA-A*2402 gene stably into T2 cell lacking TAP molecule, with or without addition of the peptide AMACR 240-248 (SEQ ID NO: 5), or with addition of a HIV-derived peptide which binds to HLA-A24; HLA-A*2402-negative K562 (ATCC strain No. CCL-243.) which is a cell line derived from chronic myelogenous leukemia and is sensitive to NK cell. The target cells were labeled with 100 μCi ⁵¹Cr for one hour. To 5×10³ target cells were added 30-fold of effector cells (T cells stimulated with a peptide) After culturing for 4 hours, the cytotoxic activity was measured. The results obtained from seven prostate cancer patients are shown in FIG. 2. In Subject 1 (Case 1), T cells stimulated with the peptide AMACR 240-248 (SEQ ID NO: 5) killed the peptide-pulsed HLA-A*2402-positive T2A24 cells, but not the cells added with the HIV-derived peptide and also HLA-A*2402-negative K562. The fact that CTLs induced by the AMACR-derived peptide AMACR 240-248 specifically killed cells presenting the AMACR-derived peptide in HLA-A*2402-restricted manner indicates that AMACR 240-248 is a tumor antigen peptide. In addition, it was demonstrated that AMACR protein from which the tumor antigen peptide was derived was a tumor antigen protein.

### EXAMPLE 4

### CTL Induction By the AMACR Antigen-Derived Peptide From Human Peripheral Blood Mononuclear Cells (2)

Using the peptide AMACR 125-133 (SEQ ID NO: 3) and AMACR 183-191 (SEQ ID NO: 4) which showed a strong binding activity to HLA-A*2402, CTLs were induced from peripheral blood mononuclear cells derived from HLA-A*2402 positive prostate cancer patients by the method as described in Example 3. The results obtained from seven prostate cancer patients are shown in FIG. 3. In two subjects (Cases 8 and 9), T cells stimulated with AMACR 125-133 or AMACR 183-191 strongly killed the peptide-pulsed HLA-A*2402 positive T2A24 cells. On the other hand, the cytotoxic activity of the T cells against other target cells such as those to which the peptide had not been added was weaker than that against the peptide-pulsed target cells.

Further, using the peptide AMACR 125-133 (SEQ ID NO: 3) and AMACR 183-191 (SEQ ID NO: 4), the cytotoxic activities of CTLs obtained in Case 9 were evaluated in different E/T (Effector (CTL) and Target (target cell)) ratios and the results are shown in FIG. 4. In a low E/T ratio (1, 3), the CTLs induced by AMACR 125-133 and AMACR 183-191 strongly killed only the peptide-pulsed HLA-A*2402 positive T2A24 cells but not the target cells used as a negative control, such as the cells to which the peptide had not been added. In a high E/T ratio (10, 30), similar to the result shown in FIG. 3, the CTLs showed a weak cytotoxic activity against negative controls including the cells without addition of the peptide but showed a strong cytotoxic activity against peptide-pulsed HLA-A*2402 positive T2A24 cells.

Those results demonstrate that, in addition to AMACR 240-248 (SEQ ID NO: 5), AMACR 125-133 (SEQ ID NO: 3) and AMACR 183-191 (SEQ ID NO: 4) are also tumor antigen peptides.

### INDUSTRIAL APPLICABILITY

The present invention provides use of AMACR and a peptide derived therefrom or a nucleic acid encoding the same and the like in the field of cancer immunotherapy. The tumor antigen protein AMACR and the AMACR-derived tumor antigen peptide of the present invention can be used to treat patients suffering from cancer such as prostate cancer.

### BRIEF DESCRIPTION OF DRAWINGS

FIG. 1 is a graph showing the binding affinities of the following peptides to HLA-A*2402: AMACR-derived peptides (four kinds); an EB virus-derived peptide (shown as EBV in the graph), an HIV virus-derived peptide (shown as HIV) (positive control); an Ovalbumin-derived peptide (SL8) (negative control). The vertical axis shows the mean fluorescence intensity (binding affinity). The horizontal axis shows names of the peptides.

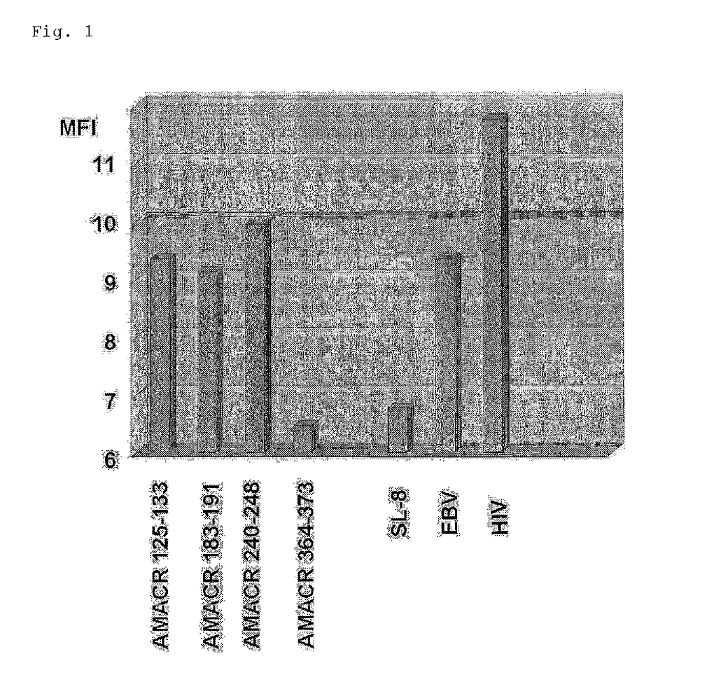

FIG. 2 shows the results of induction of a peptide-specific cytotoxic T cell (CTL) from peripheral blood lymphocytes of prostate cancer patients using the peptide AMACR 240-248. The vertical axis shows cytotoxic activity of the CTL. The horizontal axis shows the seven cases of prostate cancer (Cases 1-7). To examine the peptide specificity, the following four kinds of target cells were employed: T2A24(−), T2A24 cell only; T2A24 AMACR, T2A24 cell added with the peptide AMACR 240-248; T2A24 HIV, T2A24 cell added with an HIV peptide; K562, K562 cell only.

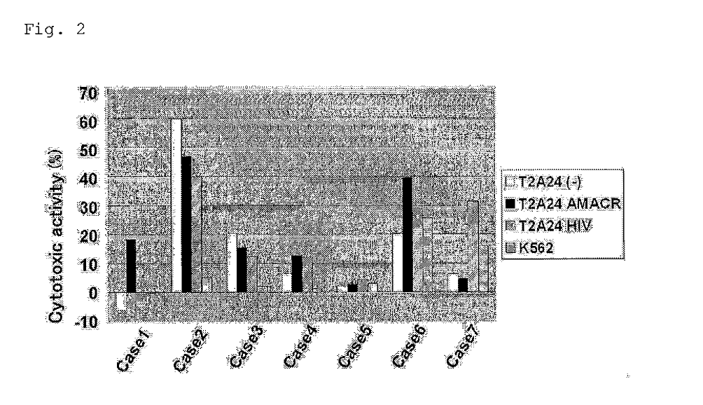

FIG. 3 shows the results of induction of a peptide-specific cytotoxic T cell (CTL) from peripheral blood lymphocytes of prostate cancer patients using the peptides AMACR 125-133 and AMACR 183-191. The vertical axis shows cytotoxic activity of the CTL. The horizontal axis shows the seven cases of prostate cancer (Cases 8-14). To examine the peptide specificity, the following four kinds of target cells were employed: T2A24(−), T2A24 cell only; T2A24 AMACR, T2A24 cell added with the peptides AMACR 125-133 and AMACR 183-191; T2A24 HIV, T2A24 cell added with an HIV peptide; K562, K562 cell only.

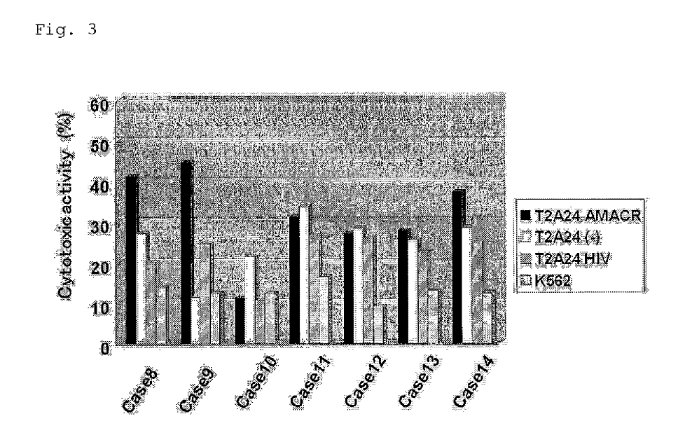

FIG. 4 shows the results of induction of a peptide-specific cytotoxic T cell (CTL) from peripheral blood lymphocytes of a prostate cancer patient (Case 9) using the peptides AMACR 125-133 and AMACR 183-191 in different E/T (Effecter (CTL))and Target (target cell)) ratios. The vertical axis shows cytotoxic activity of the CTL. The horizontal axis shows the E/T ratios (1, 3, 10, 30). To examine the peptide specificity, the following four kinds of target cells were employed: T2A24(−), T2A24 cell only; T2A24 AMACR, T2A24 cell added with the peptides AMACR 125-133 and AMACR 183-191; T2A24 HIV, T2A24 cell added with an HIV peptide; K562, K562 cell only.

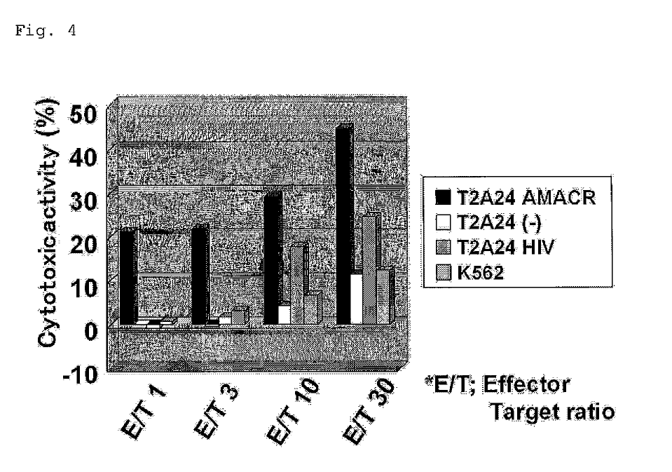

### SEQUENCE LISTING FREE TEXT

The amino acid sequences of SEQ ID NOS: 3 to 33 refer to synthetic peptides.

1. A peptide which comprises a partial peptide derived from alpha-methylacyl-CoA racemase (AMACR) and is capable of binding to an HLA antigen and is recognized by a CTL.

2. The peptide of claim 1, wherein the HLA antigen is HLA-A 24 or HLA-A2 antigen.

3. The peptide of claim 2, which comprises the amino acid sequence of any one of SEQ ID NOS: 3 to 33.

4. A peptide which comprises an amino acid sequence which is the same as the amino acid sequence of any one of SEQ ID NOS: 3 to 23 except that the amino acid at position 2 is substituted by tyrosine, phenytalanine, methionine or tryptophan, and/or the C terminal amino acid by phenylalanine, leucine, isoleucine, tryptophan or methionine, and is capable of binding to HLA-A24 antigen and is recognized by a CTL.

5. A peptide which comprises an amino acid sequence which is the same as the amino acid sequence of any one of SEQ ID NOS: 24 to 33 except that the amino acid at position 2 is substituted by leucine, methionine, valine, isoleucine or glutamine and/or the C terminal amino acid by valine or leucine, and is capable of binding to HLA-A2 antigen and is recognized by a CTL.

6. An epitope peptide which comprises the peptide of claim 1.

7. A pharmaceutical composition which comprises the peptide of claim 1 and a pharmaceutically acceptable carrier.

8. A nucleic acid which comprises a polynucleotide encoding the peptide of claim 1.

9. A pharmaceutical composition which comprises the nucleic acid of claim 8 and a pharmaceutically acceptable carrier.

10. A pharmaceutical composition which comprises AMACR and a pharmaceutically acceptable carrier.

11. The pharmaceutical composition of claim 10, wherein AMACR comprises the amino acid sequence of SEQ ID NO: 2.

12. A pharmaceutical composition which comprises a nucleic acid comprising a polynucleotide encoding AMACR and a pharmaceutically acceptable carrier.

13. The pharmaceutical composition of claim 12, wherein the polynucleotide encoding AMACR comprises the base sequence of SEQ ID NO: 1, or encodes the amino acid sequence of SEQ ID NO: 2.

14. A method of preparing an antigen presenting cell, wherein a cell having an antigen-presenting ability is brought into contact in vitro with any one of:
(a) the peptide of claim 1,
(b) a nucleic acid comprising a polynucleotide encoding the peptide of (a) above,
(c) AMACR, and
(d) a nucleic acid comprising a polynucleotide encoding AMACR.

15. An antigen presenting cell prepared by the method of claim 14.

16. A pharmaceutical composition which comprises the antigen presenting cell of claim 15 and a pharmaceutically acceptable carrier.

17. A method of inducing a CTL, wherein peripheral blood lymphocytes are brought into contact in vitro with any one of:
(a) the peptide of claim 1,
(b) a nucleic acid comprising a polynucleotide encoding the peptide of (a) above,
(c) AMACR, and
(d) a nucleic acid comprising a polynucleotide encoding AMACR.

18. A CTL induced by the method of claim 17.

19. A pharmaceutical composition which comprises the CTL of claim 18 and a pharmaceutically acceptable carrier.

20-21. (canceled)

22. An antibody which specifically binds to the peptide of claim 1.

23. An HLA monomer, HLA dimer, HLA tetramer or HLA pentamer which comprises the peptide of claim 1 and an HLA antigen.

24. A reagent for detecting a CTL specific to a AMACR-derived tumor antigen peptide, which comprises as a component the HLA monomer, HLA dimer, HLA tetramer or HLA pentamer of claim 23.


SEQUENCE LISTING (33 sequences)

SEQ ID NO: 1 - DNA, 1149 bp, Homo sapiens
  CDS (1)..(1149)
atggcactgcagggcatctcggtcgtggagctgtccggcctggccccgggcccgttctgtgctatggtcctggctgacttcggggcgcgtgtggtacgcgtggaccggcccggctcccgctacgacgtgagccgcttgggccggggcaagcgctcgctagtgctggacctgaagcagccgcggggagccgccgtgctgcggcgtctgtgcaagcggtcggatgtgctgctggagcccttccgccgcggtgtcatggagaaactccagctgggcccagagattctgcagcgggaaaatccaaggcttatttatgccaggctgagtggatttggccagtcaggaagcttctgccggttagctggccacgatatcaactatttggctttgtcaggtgttctctcaaaaattggcagaagtggtgagaatccgtatgccccgctgaatctcctggctgactttgctggtggtggccttatgtgtgcactgggcattataatggctctttttgaccgcacacgcactggcaagggtcaggtcattgatgcaaatatggtggaaggaacagcatatttaagttcttttctgtggaaaactcagaaattgagtctgtgggaagcacctcgaggacagaacatgttggatggtggagcacctttctatacgacttacaggacagcagatggggaattcatggctgttggagcaatagaaccccagttctacgagctgctgatcaaaggacttggactaaagtctgatgaacttcccaatcagatgagcatggatgattggccagaaatgaagaagaagtttgcagatgtatttgcagagaagacgaaggcagagtggtgtcaaatctttgacggcacagatgcctgtgtgactccggttctgacttttgaggaggttgttcatcatgatcacaacaaggaacggggctcgtttatcaccagtgaggagcaggacgtgagcccccgccctgcacctctgctgttaaacaccccagccatcccttctttcaaaagggatcctttcataggagaacacactgaggagatacttgaagaatttggattcagccgcgaagagatttatcagcttaactcagataaaatcattgaaagtaataaggtaaaagctagtctctaa

SEQ ID NO: 2 - protein, 382 aa, Homo sapiens
MALQGISVVELSGLAPGPFCAMVLADFGARVVRVDRPGSRYDVSRLGRGKRSLVLDLKQPRGAAVLRRLCKRSDVLLEPFRRGVMEKLQLGPEILQRENPRLIYARLSGFGQSGSFCRLAGHDINYLALSGVLSKIGRSGENPYAPLNLLADFAGGGLMCALGIIMALFDRTRTGKGQVIDANMVEGTAYLSSFLWKTQKLSLWEAPRGQNMLDGGAPFYTTYRTADGEFMAVGAIEPQFYELLIKGLGLKSDELPNQMSMDDWPEMKKKFADVFAEKTKAEWCQIFDGTDACVTPVLTFEEVVHHDHNKERGSFITSEEQDVSPRPAPLLLNTPAIPSFKRDPFIGEHTEEILEEFGFSREEIYQLNSDKIIESNKVKASL

SEQ ID NO: 3 - protein, 9 aa, Artificial
  Synthetic Peptide
NYLALSGVL

SEQ ID NO: 4 - protein, 9 aa, Artificial
  Synthetic Peptide
NMVEGTAYL

SEQ ID NO: 5 - protein, 9 aa, Artificial
  Synthetic Peptide
FYELLIKGL

SEQ ID NO: 6 - protein, 10 aa, Artificial
  Synthetic Peptide
IYQLNSDKII

SEQ ID NO: 7 - protein, 10 aa, Artificial
  Synthetic Peptide
PFCAMVLADF

SEQ ID NO: 8 - protein, 10 aa, Artificial
  Synthetic Peptide
SFCRLAGHDI

SEQ ID NO: 9 - protein, 9 aa, Artificial
  Synthetic Peptide
LMCALGIIM

SEQ ID NO: 10 - protein, 9 aa, Artificial
  Synthetic Peptide
SFLWKTQKL

SEQ ID NO: 11 - protein, 9 aa, Artificial
  Synthetic Peptide
LWKTQKLSL

SEQ ID NO: 12 - protein, 10 aa, Artificial
  Synthetic Peptide
LWEAPRGQNM

SEQ ID NO: 13 - protein, 9 aa, Artificial
  Synthetic Peptide
NMLDGGAPF

SEQ ID NO: 14 - protein, 9 aa, Artificial
  Synthetic Peptide
TYRTADGEF

SEQ ID NO: 15 - protein, 10 aa, Artificial
  Synthetic Peptide
TYRTADGEFM

SEQ ID NO: 16 - protein, 10 aa, Artificial
  Synthetic Peptide
QFYELLIKGL

SEQ ID NO: 17 - protein, 10 aa, Artificial
  Synthetic Peptide
QMSMDDWPEM

SEQ ID NO: 18 - protein, 9 aa, Artificial
  Synthetic Peptide
DWPEMKKKF

SEQ ID NO: 19 - protein, 10 aa, Artificial
  Synthetic Peptide
EMKKKFADVF

SEQ ID NO: 20 - protein, 10 aa, Artificial
  Synthetic Peptide
VFAEKTKAEW

SEQ ID NO: 21 - protein, 10 aa, Artificial
  Synthetic Peptide
PFIGEHTEEI

SEQ ID NO: 22 - protein, 10 aa, Artificial
  Synthetic Peptide
GFSREEIYQL

SEQ ID NO: 23 - protein, 9 aa, Artificial
  Synthetic Peptide
IYQLNSDKI

SEQ ID NO: 24 - protein, 9 aa, Artificial
  Synthetic Peptide
YLSSFLWKT

SEQ ID NO: 25 - protein, 9 aa, Artificial
  Synthetic Peptide
VLADFGARV

SEQ ID NO: 26 - protein, 9 aa, Artificial
  Synthetic Peptide
NMVEGTAYL

SEQ ID NO: 27 - protein, 10 aa, Artificial
  Synthetic Peptide
VLLEPFRRGV

SEQ ID NO: 28 - protein, 10 aa, Artificial
  Synthetic Peptide
FLWKTQKLSL

SEQ ID NO: 29 - protein, 10 aa, Artificial
  Synthetic Peptide
RLAGHDINYL

SEQ ID NO: 30 - protein, 10 aa, Artificial
  Synthetic Peptide
MLDGGAPFYT

SEQ ID NO: 31 - protein, 10 aa, Artificial
  Synthetic Peptide
MVLADFGARV

SEQ ID NO: 32 - protein, 10 aa, Artificial
  Synthetic Peptide
QIFDGTDACV

SEQ ID NO: 33 - protein, 10 aa, Artificial
  Synthetic Peptide
RLGRGKRSLV